(19) United States
(12) Patent Application Publication
(10) Pub. No.: US 2026/0337636 A1
(43) Pub. Date: Oct. 29, 2026

(54) CHROMATOGRAPHY METHODS FOR PURIFICATION OF AAV CAPSIDS

(71) Applicant: MeiraGTx UK II Limited, London (GB)

(72) Inventors: Jayan Senaratne, London (GB); Sian Davies, London (GB)

(21) Appl. No.: 19/117,745

(22) PCT Filed: Oct. 11, 2023
(86) PCT No.: PCT/IB2023/060237

§ 371 Date: Apr. 2, 2025

Related U.S. Application Data
(60) Provisional application No. 63/379,115, filed on Oct. 11, 2022.
(60) Provisional application No. 63/489,684, filed on Mar. 10, 2023.

(51) Int. Cl. C12N 15/864; A61K 35/76

(52) U.S. Cl. CPC C12N 15/8645; A61K 35/76; C12N 2750/14132; C12N 2750/14143; C12N 2750/14152

(57) ABSTRACT

The present disclosure provides methods for producing and purifying recombinant adeno associated virus (AAV) vectors, including separating of full capsids from empty capsids, using anion exchange chromatography in weak partitioning mode. The present disclosure also provides a population of rAAV full capsids enriched by the method, a population of rAAV full capsids isolated and enriched by the method, and a pharmaceutical composition including the populations of enriched full capsids.

### CROSS-REFERENCE TO RELATED APPLICATIONS

This application claims the benefit of priority to U.S. Provisional Application Ser. No. 63/379,115, filed Oct. 11, 2022, and to U.S. Provisional Application Ser. No. 63/489,684, filed Mar. 10, 2023, which are incorporated herein by reference in their entirety.

### FIELD

The present disclosure provides methods for producing and purifying recombinant adeno associated virus (AAV) vectors including separation of full capsids from empty capsids using anion exchange chromatography in weak partitioning mode.

### BACKGROUND

Adeno-associated virus (AAV) is a replication-deficient parvovirus. AAV particles comprise a capsid having three capsid proteins—VP1, VP2 and VP3—enclosing a single-stranded DNA genome of about 4.8 kb in length, which may be either the plus or minus strand. Particles containing either strand are infectious, and replication occurs by conversion of the parental infecting single strand to a duplex form, and subsequent amplification, from which progeny single strands are displaced and packaged into capsids.

AAV is dependent on co-infection with other viruses, mainly adenoviruses, in order to replicate. Its single-stranded genome contains three genes, rep (Replication), cap (Capsid), and aap (Assembly), which give rise to at least nine gene products through the use of three promoters, alternative translation start sites, and differential splicing. These coding sequences are flanked by inverted terminal repeats (ITRs) that are required for genome replication and packaging. The rep gene encodes four proteins (Rep78, Rep68, Rep52, and Rep40), which are involved in viral genome replication and packaging, while cap expression gives rise to the viral capsid proteins (VP1, VP2, and VP3), which form the outer capsid shell that protects the viral genome, as well as being actively involved in cell binding and internalization. The aap gene encodes the assembly-activating protein (AAP) in an alternate reading frame that overlaps the cap gene. This AAP protein is thought to provide a scaffolding function for capsid assembly.

AAV particles have features that make them attractive as vectors for therapeutic applications including gene therapy and genetics vaccines. AAV infects a wide range of cell types including many mammalian cells, allowing the possibility of targeting many different tissues in vivo. AAV infects slowly dividing and non-dividing cells. For therapeutic applications, recombinant AAV (rAAV) are used in which the genome includes a heterologous transgene and typically retains the ITRs, but lacks the viral rep, cap, and aap genes. In the absence of Rep proteins, ITR-flanked transgenes can form transcriptionally active nuclear extrachromosomal element or episome that can persist essentially for the lifetime of the transduced cells.

Important goals for the rAAV vector production method are to achieve consistent, high vector productivity while minimizing generation of product-related impurities, including AAV-encapsidated residual DNA impurities and empty capsids. Measured as vector genomes (VG) generated per cell, rAAV vector productivity can be highly variable, ranging from less than 10³ to 2×10⁵ VG per cell. In addition to higher cost-effectiveness, an important advantage of high productivity is that purification can be more efficient when the starting material has a higher ratio of the rAAV vector product to total harvest biomass.

During rAAV vector production, removal of product-related impurities is an important part of rAAV purification. Although separation of the empty capsids from the full capsids is achievable when using ultracentrifugation, scalable chromatography methods have proven more challenging. As an alternative, anion exchange chromatography in bind and elute mode has commonly been investigated in an attempt to reproduce the high full capsid ratios achieved by ultracentrifugation. However, the similar properties of the full and empty capsids lead to a sharp trade-off between product yield and full capsid ratio which makes selecting optimal conditions difficult. This is also hindered by complex interactions between the AAV capsids and chromatography matrices which often lead to further product losses. Accordingly, there is a need for rAAV purification methods that decrease product related impurities such as empty capsids.

### SUMMARY

The present invention provides methods for producing and purifying rAAV particles, including separating full capsids from empty capsids (enrichment of full capsids), using anion exchange chromatography in weak partitioning mode. Described herein is a method of enriching rAAV full capsids in a mixture of full and empty capsids, wherein the method includes:
(i) providing a solution including rAAV full and empty capsids;
(ii) equilibrating an anion exchange (AEX) column or membrane; and;
(iii) subjecting the solution including rAAV full and empty capsids to weak partition mode AEX chromatography to separate the empty capsids from the full capsids resulting in an AEX eluate enriched for full capsids.

In embodiments of the method, the solution is an affinity chromatography eluate and step (iii) includes applying the affinity chromatography eluate to the equilibrated AEX column or membrane, washing the AEX column or membrane at least once, and eluting rAAV full capsids from the AEX column or membrane. In these methods, the weak partition mode results in full capsids displacing bound empty capsids from the AEX column or membrane and the empty capsids flowing through the AEX column or membrane to produce AEX flow-through and the full capsids remaining bound to the AEX column or membrane until elution.

In some embodiments, the affinity chromatography eluate is diluted to a target salt concentration prior to being subjected to the equilibrated AEX column or membrane. The target salt concentration is typically in a range of about 85 mM to about 95 mM. The target salt concentration can be, e.g., about 90 mM and the salt can be, e.g., NaCl.

In embodiments of the method, the AEX column or membrane is equilibrated prior to loading with the solution including rAAV full and empty capsids with an equilibration buffer comprising about 10 mM to about 1000 mM (e.g., about 10, 49, 50, 51, 100, 200, 300, 400, 500, 600, 700, 800, 900, 1000 mM) Tris, about 85 mM to about 95 mM NaCl (e.g., about 84, 84.9, 85, 86, 87, 88, 89, 89.5, 90, 90.5, 91, 92, 93, 94, 95, 95.5), and greater than about 0.5% (e.g., 0.5, 0.6, 0.7, 0.75, 0.8, 0.9, 1.0%) (w/v) polysorbate 80, wherein the equilibration buffer is at a pH of about 9.0 (e.g., about 8.8, 8.9, 9.0, 9.1, 9.2). The equilibration buffer can include about 50 mM Tris, about 90 mM NaCl, and about 0.75% polysorbate 80, and have a pH of about 9.0.

In embodiments of the method, washing the AEX column or membrane at least once includes a first wash buffer including (i) about 10 mM to about 1000 mM (e.g., about 10, 49, 50, 51, 100, 200, 300, 400, 500, 600, 700, 800, 900, 1000 mM) Tris; (ii) about 85 mM to about 95 mM (e.g., about 84, 84.9, 85, 86, 87, 88, 89, 89.5, 90, 90.5, 91, 92, 93, 94, 95, 95.5) NaCl, and (iii) greater than about 0.5% (e.g., 0.5, 0.6, 0.7, 0.75, 0.8, 0.9, 1.0%) (w/v) polysorbate 80, wherein the first wash buffer is at about pH 9.0 (e.g., about 8.8, 8.9, 9.0, 9.1, 9.2). The first wash buffer can include e.g., (i) about 50 mM tris; (ii) about 90 mM NaCl, and (iii) about 0.75% polysorbate 80, and be at about pH 9.0.

In embodiments, washing the AEX column or membrane at least once further includes a second wash with a second wash buffer including (i) about 10 mM to about 1000 mM (e.g., about 10, 49, 50, 51, 100, 200, 300, 400, 500, 600, 700, 800, 900, 1000 mM) tris; (ii) about 100 mM to about 125 mM (e.g., about 99, 100, 101, 105, 110, 120, 124, 125, 126 mM) NaCl, and (iii) greater than about 0.5% (e.g., 0.5, 0.6, 0.7, 0.75, 0.8, 0.9, 1.0%) (w/v) polysorbate 80, wherein the second wash buffer is at about pH 9.0 (e.g., about 8.8, 8.9, 9.0, 9.1, 9.2). The second wash buffer can include e.g., (i) about 50 mM tris; (ii) about 125 mM NaCl, and (iii) about 0.75% polysorbate 80, and be at about pH 9.0.

In embodiments, the full capsids are eluted from the AEX column or membrane with an elution buffer including (i) about 10 mM to about 1000 mM (e.g., about 10, 49, 50, 51, 100, 200, 300, 400, 500, 600, 700, 800, 900, 1000 mM) tris; (ii) about 125 mM to about 250 mM (e.g., about 124, 125, 126, 130, 140, 150, 160, 170, 180, 190, 200, 210, 220, 230, 240, 250, 251 mM) NaCl, and (iii) greater than about 0.5% (e.g., 0.5, 0.6, 0.7, 0.75, 0.8, 0.9, 1.0%) (w/v) polysorbate 80, wherein the elution buffer is at about pH 9.0 (e.g., about 8.8, 8.9, 9.0, 9.1, 9.2). The elution buffer can include e.g., (i) about 50 mM tris; (ii) about 250 mM NaCl, and (iii) about 0.75% polysorbate 80, wherein the elution buffer is at a pH of about 9.0.

In embodiments of the method, the AEX chromatography column or membrane includes a high flow rate adsorptive membrane, e.g., Sartobind® Q chromatography membranes. In embodiments, the method can further include the step of subjecting the AEX eluate including enriched full capsids to tangential flow filtration (TFF) resulting in a purified preparation of full capsids (e.g., a drug substance).

In embodiments, Polysorbate 20 and poloxamer 188 can be used in one or more of the buffers described herein.

In embodiments of the method, the method further includes the step of subjecting the AEX eluate to analytical ultracentrifugation to quantify full capsid enrichment.

In embodiments of the method, the enriched full capsids can include AAV serotype 2 capsid proteins and a polynucleotide sequence comprising a transgene, e.g., a transgene such as aquaporin 1 (AQP1).

In embodiments, the enriched full capsids include greater than 80% full capsids, (e.g., about 80.5, 81.5, 82, 83, 84, 85, 86, 87, 88, 89, 90, 91, 92, 93, 94, 95, 96% full capsids), greater than 90% full capsids, and greater than 95% full capsids.

Also described herein is a method of isolating and enriching rAAV full capsids in a mixture of full and empty capsids. The method includes: isolating a mixture of rAAV full and empty capsids from viral production cells by lysing the cells and clarifying the resulting lysate; subjecting the clarified lysate to affinity chromatography resulting in an affinity chromatography eluate; and subjecting the affinity chromatography eluate to weak partition mode AEX chromatography to separate empty capsids from full capsids resulting in an AEX eluate comprising enriched full capsids. In embodiments, the cells are mammalian cells. The cells are typically cultured in suspension, e.g., in a shaker flask, a spinner flask, a cellbag or a bioreactor.

The present invention also provides a population of rAAV full capsids enriched by the method, a population of rAAV full capsids isolated and enriched by the method, and a pharmaceutical composition including the populations of enriched full capsids.

### BRIEF DESCRIPTION OF THE FIGURES

FIG. 1 is a diagram showing the charge of empty and full AAV capsids above and below the pI (1).

FIG. 2 is a diagram showing the weak partitioning process, where the empty capsids are replaced by full capsids due to the environment created.

FIG. 3 is a graph showing the relationship between wash 2 and eq./feed NaCl concentrations (mM), and the empty capsid removal (VP/mL).

FIG. 4 is a graph showing the relationship between wash 2 and eq./feed NaCl concentrations (mM), and the full capsid ratio (%) in the elution.

FIG. 5 is a graph showing the relationship between wash 2 and eq./feed NaCl concentrations (mM), and the VG recovery (%) in the elution.

FIG. 6 is a plot showing the optimal region based on the wash 2 and eq./feed NaCl concentrations (mM).

FIG. 7 is a graph showing the relationship between load ratio, and full capsid (VG) and empty capsid (VP) breakthrough (%).

FIG. 8 is a chromatogram of an optimized AAV-AQP1 AEX step at 2 Liter (L) scale.

FIG. 9 is a graph showing a summary of results demonstrating the scalability of the AEX process. The 2 L scale refers to the process operated using a 10 mL Sartobind® Q and the 80 refers to the process operated using a 75 mL Sartobind® Q.

FIG. 10 is a graph showing results of the breakthrough experiment showing the extent of breakthrough in the flow-through for vector genomes and vector particles.

FIG. 11 is an excerpt of the chromatogram from the experiment (described in Example 2) performed to confirm the process using a 10 mL Sartobind® Q. This excerpt focusses on the column wash 2 and elution phases.

FIG. 12 shows results of the analytical ultracentrifugation performed on the eluate produced by the confirmation run performed on a 10 mL Sartobind® Q.

FIG. 13 is a chromatogram of the initial anion exchange chromatography run through.

FIG. 14 shows a breakthrough curve indicating both the VG breakthrough and empty capsid breakthrough up to a load challenge of 4E14 VG/mL at 90 mM NaCl feed and equilibration conductivity.

FIG. 15 is a pair of graphs showing the VG concentration on the column as the VG load challenge is increased to 4E14 vg/ml (top graph) and the empty capsid ratio on the column as the VG load challenge is increased to 4E14 VG/mL (bottom graph).

FIG. 16 is a graph showing the increase in full capsid ratio on the column as VG load challenge increases to 4E14 VG/mL

FIG. 17 is a graph showing the concentration of full capsids on the column when loading to 4E14 VG/mL at both 9 mS/cm and 8 mS/cm to highlight the level of enrichment achievable.

FIG. 18 is a chromatogram of the optimized AAV-AQP1 AEX step at 20 L scale confirmation run.

FIG. 19 shows the VG recovery and full capsid % values after each of: USP, capture chromatography, ion exchange chromatography (IEX), and tangential flow filtration (TFF) in the “Initial Process” and “Final Process”.

FIG. 20 is a graph showing a comparison between the full capsid ratio and VG recovery of a 20 L and 80 L batch and a plot showing an analytical ultracentrifugation (AUC) result confirming the elution peak in FIG. 8 contains 82% full capsids.

### DETAILED DESCRIPTION

The present disclosure provides methods for producing and purifying rAAV, including separating full capsids from empty capsids (enrichment of full capsids), using anion exchange chromatography in weak partitioning mode. The methods of enriching rAAV full capsids in a mixture of full and empty capsids include the following steps:
(i) providing a solution including rAAV full and empty capsids;
(ii) equilibrating an anion exchange (AEX) column or membrane; and
(iii) subjecting the solution including rAAV full and empty capsids to weak partition mode AEX chromatography to separate the empty capsids from the full capsids resulting in an AEX eluate enriched for full capsids.

Methods of isolating and enriching rAAV full capsids in a mixture of full and empty capsids include the following steps:
(i) isolating a mixture of rAAV full and empty capsids from viral production cells by lysing the cells and clarifying the resulting lysate;
(ii) subjecting the clarified lysate to affinity chromatography resulting in an affinity chromatography eluate; and
(iii) subjecting the affinity chromatography eluate to weak partition mode AEX chromatography to separate empty capsids from full capsids resulting in an AEX eluate including enriched full capsids.

The present invention also provides a population of rAAV full capsids enriched by the method, a population of rAAV full capsids isolated and enriched by the method, and pharmaceutical compositions (e.g., drugs, drug substances) including the populations of enriched full capsids. In embodiments, the methods provide increased production titers and higher ratio of full to empty capsids (F:E). More specifically, the disclosure provides, e.g., a method of enriching rAAV full capsids in a mixture of full and empty capsids. In embodiments, the production yield is greater than 50%, greater than 50% of the empty capsids are removed, and a F:E ratio of greater than 80% is achieved.

AEX in Weak Partitioning Mode for Purifying rAAV

The purification and enrichment methods described herein involve purifying rAAV full capsids (i.e., rAAV particles containing a recombinant genome) from a solution containing rAAV full and empty capsids. The solution can be the result of any rAAV production/purification method. Typically, the solution is conditioned by diluting to a target salt (e.g., NaCl) concentration before being subjected to AEX chromatography. For example, the solution can be diluted to a NaCl concentration of about 90 mM (e.g., about 89, 90, 91 mM) with about 10 mM to about 1000 mM (e.g., about 10, 49, 50, 51, 100, 200, 300, 400, 500, 600, 700, 800, 900, 1000 mM) Tris, about 0.75% (w/v) polysorbate 80, and a pH of about 9.0 (e.g., about 8.8, 8.9, 9.0, 9.1, 9.2). In embodiments, the solution is an affinity chromatography eluate. Affinity chromatography is one of several purification steps in a typical method of producing and purifying rAAV particles (e.g., rAAV full capsids). Methods of rAAV production/purification are well known in the art and examples of such methods are described below.

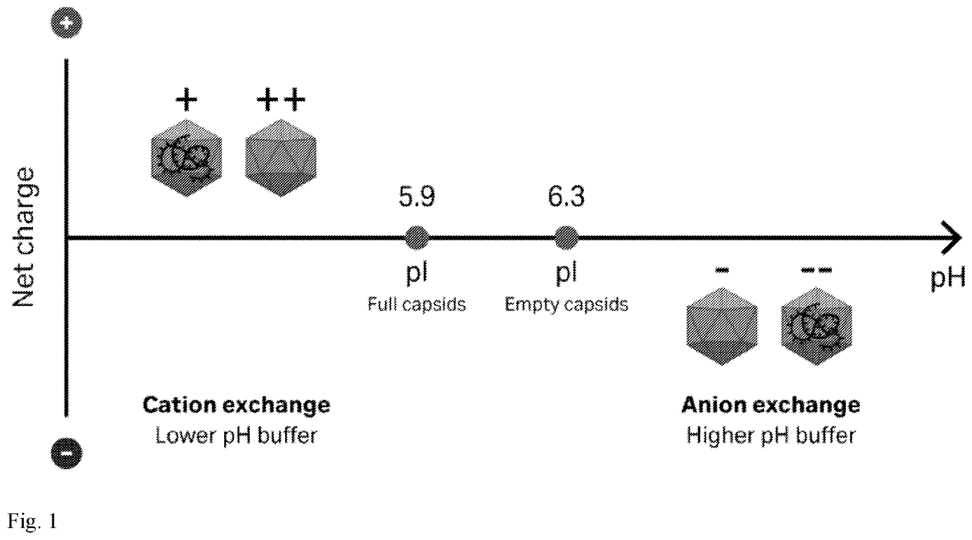

In the methods described herein, weak partition mode AEX chromatography is a purification technique used to separate the full capsids from the empty capsids (FIGS. 1 & 2). This form of chromatography exploits the charge difference between capsids containing viral genome and those that are empty. Targeting pH values above the capsid pI means the VG-containing full capsids carry a greater negative charge than the empty capsids. In the purification and enrichment methods described herein, weak partitioning describes the process of separating full capsids from empty capsids by creating an environment in which the binding of full capsids is more favorable.

Anion exchange chromatography in weak partitioning mode is distinct from historically used flow-through mode AEX and bind-and-elute mode AEX methods (Liu et al., MAbs. 2010; 2 (5): 480-499; Kelley et al., Biotechnology and Bioengineering vol. 101:553-566, 2008). In bind-and-elute mode AEX, the product (of interest) pool is first loaded onto an anion exchange column and the product of interest is then eluted with a higher salt concentration in a step or linear gradient, leaving the majority of impurities bound to the column. The impurities are eluted from the column during the cleaning or regeneration step. In flow-through mode AEX, the operating pH is normally 8 to 8.2, with a conductivity of up to 10 mS/cm in the product load and equilibration and wash buffers. Conditions are chosen such that the product does not bind to the column, while acidic impurities such as nucleic acid and host cell proteins do. Use of anion exchange chromatography in weak partitioning mode can enable a two chromatography recovery process comprising affinity chromatography and anion exchange for products of interest. As with flow-through chromatography, the process is run isocratically, but, in contrast to flow-through mode, the conductivity and pH are chosen such that the binding of both the product and impurities are enhanced, attaining a product partition coefficient (Kp) between 0.1-20, and preferably between 1 and 3. This takes advantage of the fact that the impurities to be removed are more acidic than the product. Both product and impurities bind to the anion exchange resin, but the impurities are much more tightly bound than in flow-through mode, which can lead to an increase in impurity removal. Thus, weaker binding impurities that are not removed efficiently in flow-through mode can be removed to a greater degree under conditions where their partition coefficient (Kp) has been increased. Due to the increased clearance of virus, host cell protein and product related species compared to anion exchange chromatography in flow-through mode, weak partitioning chromatography can enable a two column recovery process. One aspect of weak partitioning chromatography is that the pH and counterion conditions need to be optimized for each product. This is in contrast to some platform chromatography processes that are able to use standardized conditions on an anion exchange matrix (resin or membrane) for most products.

In the methods of enriching rAAV full capsids in a mixture of full and empty capsids, a solution including rAAV full and empty capsids is provided. As discussed above, the solution can be the result of any rAAV production/purification method and in embodiments, the solution is an affinity chromatography eluate. An AEX column or membrane that will receive the solution is typically pre-equilibrated with a suitable pre-equilibration buffer. For example, a suitable pre-equilibration buffer can include about 50 mM Tris, about 1 M NaCl, and about 0.75% polysorbate 80 and have a pH of about 9.0 (e.g., about 8.8, 8.9, 9.0, 9.1, 9.2). After the pre-equilibration step, the AEX column or membrane is equilibrated with an equilibration buffer. Typically, the equilibration buffer includes about 10 mM to about 1000 mM (e.g., about 10, 49, 50, 51, 100, 200, 300, 400, 500, 600, 700, 800, 900, 1000 mM) Tris, about 85 mM to about 95 mM (e.g., about 84, 84.9, 85, 86, 87, 88, 89, 89.5, 90, 90.5, 91, 92, 93, 94, 95, 95.5) salt (e.g., NaCl), and greater than about 0.5% (e.g., 0.5, 0.6, 0.7, 0.75, 0.8, 0.9, 1.0%) (w/v) polysorbate 80. In some embodiments, the equilibration buffer includes about 50 mM Tris, about 90 mM NaCl, and greater than 0.5% (w/v) polysorbate 80, and about 0.75% polysorbate 80, and has a pH of about 9.0. In the methods, any suitable AEX column(s) or membrane(s) can be used. In embodiments, a high flow rate adsorptive membrane is used. An example of such a membrane is the Sartobind® Q chromatography membrane. Another example of such a membrane is the Pall Corporation's Mustang® Q membrane.

After equilibration of the AEX column or membrane, the solution (referred to as “feed”, “feed material” and “conditioned feed” in Examples 1 and 2) is applied to the equilibrated AEX column or membrane. The solution can be applied at any suitable load ratio. In the experiments described in Examples 1 and 2, a load ratio of 5×10¹³-1×10¹⁵ VG/mL was used and resulted in a full capsid ratio elution % of 81.5%. The loaded solution flow-through typically consists of empty capsids (full capsids will have stronger interactions with the column or membrane and displace empty capsids). In embodiments, the flow-through or a sample thereof can be analyzed to determine its viral genome and viral particle concentrations by any suitable methods.

Subsequent to the solution application, the AEX column or membrane is washed at least once (e.g., once, twice, three times). In the at least first wash, the AEX column or membrane is washed with a first wash buffer including about 10 mM to about 1000 mM (e.g., about 10, 49, 50, 51, 100, 200, 300, 400, 500, 600, 700, 800, 900, 1000 mM) Tris, about 85 mM to about 95 mM (e.g., about 84.5, 84.9, 85, 85.5, 86, 87, 88, 89, 90, 91, 92, 93, 94, 95, 95.5, 96 mM) NaCl, and greater than about 0.5% (e.g., about 0.5, 0.6, 0.7, 0.75, 0.8, 0.9, 1.0%) (w/v) polysorbate 80. The first wash buffer is typically at about pH 9.0 (e.g., about 8.8, 8.9, 9.0, 9.1, 9.2). For example, a first wash buffer can include about 50 mM tris, about 90 mM NaCl, and about 0.75% polysorbate 80, and have a pH of about 9.0. In embodiments, there is a second wash with a second wash buffer including about 10 mM to about 1000 mM (e.g., about 10, 49, 50, 51, 100, 200, 300, 400, 500, 600, 700, 800, 900, 1000 mM) Tris, about 100 mM to about 125 mM NaCl (e.g., about 99, 100, 101, 105, 110, 120, 124, 125, 126 mM) NaCl, and greater than about 0.5% (e.g., 0.5, 0.6, 0.7, 0.75, 0.8, 0.9, 1.0%) (w/v) polysorbate 80. The second wash buffer is typically at a pH of about 9.0 (e.g., about 8.8, 8.9, 9.0, 9.1, 9.2). For example, a second wash buffer can include about 50 mM tris, about 125 mM NaCl, and about 0.75% polysorbate 80, the second wash buffer having a pH of about 9.0. The column or membrane washes typically consist mostly of empty capsids but may contain full capsids. The washes, or fractions (samples) of the washes, can be analyzed for their viral genome and viral particle concentrations by any suitable method.

Subsequent to the at least one wash (e.g., two washes), full capsids are eluted from the column or membrane resulting in an AEX eluate enriched for full capsids. Full capsids are generally eluted from the AEX column or membrane with an elution buffer including about 10 mM to about 1000 mM (e.g., about 10, 49, 50, 51, 100, 200, 300, 400, 500, 600, 700, 800, 900, 1000 mM) Tris, about 125 mM to about 250 mM (e.g., about 124, 125, 126, 130, 140, 150, 160, 170, 180, 190, 200, 210, 220, 230, 240, 250, 251 mM) NaCl, and greater than about 0.5% (e.g., 0.5, 0.6, 0.7, 0.75, 0.8, 0.9, 1.0%) (w/v) polysorbate 80. The elution buffer is typically at about pH 9.0 (e.g., about 8.8, 8.9, 9.0, 9.1, 9.2). An example of an elution buffer is a buffer that includes about 50 mM tris, about 250 mM NaCl, and about 0.75% polysorbate 80, and having a pH of about 9.0. A sample of the AEX eluate enriched for full capsids can be analyzed for its viral genome and viral particle concentrations, as well as its full capsid ratio and viral genome recovery. Accordingly, in embodiments, the methods can further include subjecting the AEX eluate enriched for full capsids or a sample thereof to pPCR and analytical ultracentrifugation (AUC) to quantify full capsid enrichment (e.g., monitoring the quality and efficacy of vector purification, measuring the relative amount of empty capsids in a preparation of recombinant viral particles). AUC is a broadly applicable and information-rich method for investigating macromolecular characteristics such as size, shape, stoichiometry, and binding properties, all in the true solution-state environment. AUC can assess quantitative and qualitative information at moderately high concentrations. Methods of characterizing preparations of recombinant viral particles using AUC are known (see, e.g., US Patent Pub. No. 20200225139 incorporated by reference herein).

For all of the pre-equilibration, equilibration, wash and elution steps, any suitable membrane volumes (MV) and flow rates (MV/min) can be used. Tables 6 and 8 show examples of suitable MV and MV/min. Specific examples of methods of enriching rAAV full capsids in a mixture of full and empty capsids are described in Examples 1 and 2.

In embodiments, the AEX eluate enriched for full capsids is subjected to TFF (e.g., TFF dialysis) for additional purification and preparation of a formulation (e.g., drug, drug substance) that can be administered as a gene therapy.

In embodiments, the method provides a rAAV full particle (full capsid) to empty AAV particle (empty capsid) ratio of at least about 30%, e.g., about 30%-40%, at least 65%, about 65%-95%, at least 80%, about 80%-85%, (e.g., 79%, 79.5%, 80%, 80.5%, 81%, 81.5%, 82%, 83%, 85%, 85%), about 90%-95% (e.g., 89%, 90%, 91%, 92%, 93%, 94%, 95%, 95.5%) etc. Levels of nuclease-resistant, AAV-encapsidated DNA impurities can be assessed by qPCR using primers and probes designed for relevant sequences in helper plasmids, or to high copy number genomic sequences. Sensitivity to nuclease treatment performed prior to qPCR allows distinction between nuclease-sensitive ‘naked’ residual DNA impurities and nuclease-insensitive-encapsidated residual DNA impurities. Total AAV capsids can be measured using capsid-specific ELISA assays and the amount of empty capsid determined by comparison of the capsid particle titer and the VG titer. Spectrophotometric methods can be used for samples in which non-AAV capsid impurities have been substantially removed.

The methods are scalable to manufacturing scale, for example, cultures of about 5 to about 10, about 10 to about 20, about 20 to about 50, about 50 to about 100 (e.g., 79, 80, 81), about 100 to about 200 or more liters, and are applicable to rAAV comprising a wide variety of AAV serotypes/capsid variants. The experiments described in Examples 1 and 2 demonstrate that rAAV full capsids can be enriched on a large (manufacturing) scale.

rAAV vectors (rAAV full capsids) produced, isolated, purified and enriched by the methods disclosed herein are useful for expressing a transgene in a target cell. These rAAV vectors may be used in gene therapy as they can introduce into a target cell a polynucleotide comprising a transgene that may be maintained and expressed in target cells. rAAV vectors are able to deliver heterologous polynucleotide sequences (e.g., polynucleotide sequences encoding a therapeutic protein or a reporter protein and regulatory elements for expression of the protein) to target cells in human patients. A non-exhaustive list of examples of transgenes includes RPGR, RPE65, GAD65, GAD67, CNGB3, and AQP1. In some embodiments, the two AAV ITRs are AAV2 ITRs. In the method, the AAV cap gene can be from an AAV serotype or AAV variant such as, for example, AAV1, AAV2, AAV3, AAV4, AAV5, AAV6, AAV7, AAV8, AAV9, AAV10, AAV 11, AAV12, AAV13, AAVrh10, AAV-PHP.5, AAV-PHP.B, AAV-PHP.eB, AAV2-retro, AAV9-retro, AAVrh74, AAVrh, and a hybrid thereof.

The term “vector” refers to a vehicle for introducing a polynucleotide into a target cell. Vectors can be viral vectors (e.g., rAAV vector, HSV vector) or non-viral vectors such as plasmids, or DNA associated with compounds such as liposomes, gelatin, or polyamines. An expression vector is a vector that contains a polynucleotide sequence encoding a gene product (e.g., a protein or RNA) with the regulatory elements for expression in a host or target cell.

A “rAAV”, “rAAV vector”, “rAAV particle” or “rAAV virion” refer to a recombinant AAV vector genome packaged in (i.e., encapsidated by) capsid proteins for subsequent infection of a target cell, ex vivo, in vitro, or in vivo. These phrases exclude empty AAV capsids and AAV capsids lacking full recombinant AAV genome containing the transgene to be expressed in the target cell. Thus, a rAAV vector, in addition to the capsid, comprises a rAAV genome. A “rAAV genome” or “rAAV vector genome” refers to the polynucleotide sequence containing a transgene of interest that is ultimately packaged or encapsidated to form a rAAV particle. Typically, for rAAV, most of the AAV genome (including, e.g., the rep, cap, and aap genes) has been deleted, with one or both ITR sequences remaining as part of the rAAV genome along with the transgene. “Transgene” as used herein refers to a polynucleotide sequence encoding a gene product (for example, a therapeutic protein or reporter protein) and regulatory elements for expression of the gene product in a target cell.

“Empty capsids” and “empty particles” refer to AAV particles having an AAV capsid shell, but lacking in whole, or in part, the recombinant AAV genome comprising the transgene sequence and one or two ITRs. Such empty capsids do not function to transfer the transgene into a target cell or cells. In embodiments, the isolated rAAV particles are separated from empty AAV particles.

The rAAV genome (including, e.g., the ITRs) can be based on the same strain or serotype (or subgroup or variant), or they can be different from each other. As a non-limiting example, a rAAV plasmid or vector genome or particle (capsid) based upon one serotype genome can be identical to one or more of the capsid proteins that package the vector genome. In addition, a rAAV genome can be derived from an AAV genome (e.g., comprise one or more ITRs derived from the AAV2 genome) that are distinct from one or more of the capsid proteins that package the rAAV vector genome.

rAAV vectors (rAAV full capsids) that can be produced, isolated, purified and enriched by the methods disclosed herein include any rAAV vectors comprising capsids and genomes derived from any AAV strain or serotype. As non-limiting examples, a rAAV vector capsid and/or genome can be based upon AAV1, AAV2, AAV3, AAV4, AAV5, AAV6, AAV7, AAV8, AAV9, AAV10, AAV11, AAV12, AAV13, AAV-PHP-5, AAV-PHP-B, AAV-PHP-eB, AAV2-retro, AAV9-retro, AAVrh74, AAVrh, AAVrh.10 (i.e., an AAV containing AAVrh.10 ITRs and AAVrh.10 capsid proteins), etc. In embodiments, the rAAV vector comprises a genome and capsid proteins derived from the same AAV strain or serotype. For example, the rAAV vector can be an rAAV2 vector (i.e., an rAAV containing AAV2 ITRs and AAV2 capsid proteins).

In embodiments, the AAV vector is a pseudotyped rAAV vector, containing ITRs from one AAV serotype and capsid proteins from a different AAV serotype. In some embodiments, the pseudotyped rAAV is rAAV2/5 (i.e., an rAAV containing AAV2 ITRs and AAV5 capsid proteins); rAAV2/8 (i.e., an rAAV containing AAV2 ITRs and AAV8 capsid proteins); rAAV2/9 (i.e., an AAV containing AAV2 ITRs and AAV9 capsid proteins) rAAV2/10 (i.e., an rAAV containing AAV2 ITRs and AAV10 capsid proteins). In embodiments, the rAAV vector comprises a capsid protein that is a variant AAV capsid such as the AAV2 variant rAAV2-retro (SEQ ID NO:44 from WO 2017/218842, incorporated herein by reference).

Methods of Producing rAAV

As explained above, the rAAV purification and enrichment methods can be applied to a solution (e.g., lysate, eluate) containing rAAV full and empty capsids that is obtained or produced by any suitable production methods. Methods of producing rAAV are well known in the art. Generally, the methods include expanding producer cells, introducing into the producer cells rAAV vector, AAV rep and cap, and helper gene nucleic acid sequences, culturing the transduced producer cells under conditions such that rAAV particles are produced, and isolating the rAAV particles. Specific embodiments of methods of producing rAAV are described in detail below.

### Cell Lines

rAAV vector production methods generally require certain elements including, for example: (i) a permissive host cell for rAAV production (producer cell); (ii) helper virus functions which can be supplied, e.g., by a suitable construct containing genes providing adenoviral helper functions; (iii) a trans-packaging rep/cap construct; and (iv) suitable production media.

A producer cell is any cell that is a permissive host cell for production of rAAV once the rAAV genome production construct, helper function construct, and construct providing AAV functions (e.g., expressing rep and cap) are present. The term can also include the progeny of the original cell which has been transfected. Thus, a producer cell is also a host cell which has been transfected with exogenous DNA sequence, or the progeny of the host cell where that DNA sequence has integrated into the host cell genome. It is understood that the progeny of a single parental cell may not necessarily be completely identical in morphology or in genomic or total DNA complement as the original parent, due to natural, accidental, or deliberate mutation.

In embodiments, cells used to produce rAAV particles are mammalian cells, including HEK293 cells, BHK cells, and HeLa cells. Exemplary producer/host cells include human embryonic kidney (HEK) cells such as HEK293. In preferred embodiments, the producer cells are adapted for growth in suspension, including suspension adapted HEK293 cells. In further preferred embodiments, the producer cells are adapted for growth in serum-free medium. In embodiments, the producer cells are increased in at least one culture vessel which can be one or more of, for example, a shaker flask, a spinner flask, a cellbag or a bioreactor.

Producer cell lines that can be used in the rAAV production, isolation, purification and enrichment methods disclosed herein include mammalian or insect cell lines. The term “cell line” refers to a population of cells capable of continuous or prolonged growth and division in vitro under appropriate culture conditions. Cell lines can, but need not be, clonal populations derived from a single progenitor cell. In cell lines, spontaneous or induced changes can occur in karyotype during storage or transfer of such clonal populations, as well as during prolonged passaging in tissue culture. Thus, progeny cells derived from the cell line may not be precisely identical to the ancestral cells or cultures.

For rAAV production to occur, the producer cell line may require that one or more of the rAAV genome production construct, helper function construct, and/or AAV rep/cap construct are present within the producer cell. These may be introduced as three constructs (e.g., three plasmids) or the producer cells may already have one or more constructs providing some or all of these functions stably integrated into the producer cell genome. In embodiments, one or more helper genes can include all or part of one or more adenovirus genes, herpes simplex virus type 1 genes, or baculovirus genes. As used herein, the term “stable” in reference to a cell, or “stably integrated” means that the nucleic acid sequences, such as a selectable marker and/or heterologous nucleic acid sequence, or plasmid or vector (or portion thereof) has been inserted into a chromosome (e.g., by homologous recombination, non-homologous end joining, transfection, etc.) or is maintained in the recipient cell or host organism extrachromosomally, and has remained in the chromosome or is maintained extrachromosomally for a period of time.

### Expanding the Producer Cell Line

In embodiments of the method described herein, an expansion phase or expansion step is used to increase the number of producer cells prior to the step of introducing the rAAV genome production construct and/or other constructs providing helper virus functions and AAV functions. The expansion phase or expansion step may be performed in one or more cell culture vessels. For example, the expansion phase or expansion step may be performed in a series of cell culture vessels of increasing volume. The cell culture medium used for expansion of the producer cell line can be any medium appropriate for the growth (i.e., increase in number) of the producer cells. In preferred embodiments, the expansion phase culture media is animal component free, and does not include, for example serum or other components derived from animals. Chemically defined, animal component-free media is commercially available.

In embodiments, an anticlumping supplement, at times referred to herein as anti-clumping agent (ACA), is added to the expansion medium to reduce cell aggregation. ACA is commercially available from, e.g., Irvine Scientific. The anti-clumping supplement may be added at one or more time points to the expansion phase culture media. In embodiments, the anti-clumping supplement comprises dextran sulfate, heparin and/or other sulfated glycosaminoglycans that suppress the aggregation of the producer cells. In embodiments, the anticlumping supplement comprises sodium heparin, which can be added to the media to concentrations of about 25 μg/ml to about 250 μg/ml, for example, about 25 μg/ml, about 50 μg/ml, about 100 μg/ml, about 150 μg/ml, and/or about 200 μg/ml.

In embodiments, the expansion phase culture media comprises, and/or is supplemented to comprise, one or more of glutamine, a glutamine precursor or an amino acid dipeptide including glutamine at concentrations from about 2 mM to about 6 mM (e.g., about 2 mM, about 3 mM, about 4 mM, about 5 mM or about 6 mM). The one or more of glutamine, glutamine precursor or an amino acid dipeptide including glutamine can be one or more of, e.g., L-alanyl-L-glutamine, L-glutamine, glutamate, glycyl-L-glutamine, glutamine protein hydrolysate, L-glutamic acid, and a glutamine dipeptide. A commercially available example of a glutamine supplement provided as the dipeptide L-alanyl-L-glutamine is GlutaMAX (ThermoFisher).

In embodiments, the expansion phase culture media comprises, and/or is supplemented to comprise, a non-ionic polyol surface-active agent such as poloxamer 188 (a copolymer of polyethylene and polypropylene ether glycol). In embodiments, the non-ionic polyol surface-active agent is present in the expansion phase culture media at about 0.05% to about 0.2% (w:v) (e.g., about 0.05%, about 0.1%, about 0.1%, or about 0.2%). In embodiments, the expansion phase culture media comprises about 4 mM L-alanyl-L-glutamine dipeptide and 0.1% (w:v) poloxamer 188.

In embodiments, the pH of the expansion phase culture media is maintained at a pH of about 7.1 to about 7.5 (e.g., about 7.1, about 7.2, about 7.3, about 7.4, or about 7.5). In embodiments, the pH is maintained at about 7.2 to about 7.4 by CO₂ sparging. In embodiments, prior to introducing into the cells one or more polynucleotide constructs, the pH of the culture medium is changed to about 6.9 and CO₂ sparging is halted.

### Introducing One or More Polynucleotide Constructs

rAAV vector generation typically requires a production cell line that provides the basic biosynthetic machinery, as well as (i) a construct that provides the rAAV genome (the transgene of interest and associated regulatory elements flanked by AAV ITRs) and (ii) one or more constructs with additional genes that provide the gene products needed to direct rAAV vector production. These additional genes include AAV-derived genes (e.g., AAV rep and cap) and helper virus-derived genes (e.g., adenovirus E1a, E1b, E2a, E4 and VA) required to support vector genome replication and packaging.

“Helper virus genes” or “helper virus-derived genes” refers to non-AAV derived viral genes whose gene products AAV is dependent on for replication. The term includes proteins and/or RNAs that are required in AAV replication, including those involved in activation of AAV gene transcription, stage specific AAV mRNA splicing, and AAV DNA replication. Helper virus genes can be derived from any of the known AAV helper viruses such as adenovirus, herpesvirus and vaccinia virus. Thus, “helper virus functions” refers to those functions provided by helper virus genes that are required for AAV production (e.g., adenovirus E1a, E1b, E2a, E4 and VA). These helper virus functions can be provided on one or more vectors introduced into the producer cells, stably expressed by the producer cells, or a combination of both.

As used herein, “AAV functions,” or “AAV accessory functions” refer to AAV-derived coding sequences which can be expressed in producer cells to provide AAV gene products that function in trans for productive AAV replication and packaging. Thus, AAV functions include AAV open reading frames (ORFs), including rep and cap and others such as aap for certain AAV serotypes. Such AAV functions are provided by one or more polynucleotide constructs, which can be plasmid vectors, non-plasmid vectors, or a polynucleotide construct that has been integrated into a chromosome of the producer cell, that provides AAV helper functions. Plasmids that provide AAV functions that may be used in the methods disclosed herein are commercially available.

In embodiments of the method, one or more of the helper virus genes are constitutively expressed by producer cells (e.g., HEK293 cells), while other helper virus genes are introduced into the producer cells, e.g., by transfection of one or more polynucleotide constructs encoding the remaining helper virus genes needed for AAV production. The AAV-derived genes (e.g., rep and cap) may be included in the same polynucleotide construct containing one or more helper virus genes, or may be on a separate polynucleotide construct. rAAV particles are produced after a polynucleotide construct providing or encoding the rAAV genome (e.g., a rAAV genome production construct) is introduced into the producer cell line. In embodiments, the rAAV particles are produced after transiently transfecting producer cells with (i) an rAAV genome production vector, and (ii) one or more vectors that provide helper virus genes (e.g., E4, E2a, and VA) and AAV genes (e.g., rep and cap). In embodiments these vectors are plasmids.

In embodiments of the method disclosed herein, following the expansion phase a first polynucleotide construct comprising a transgene flanked by ITRs, and a second polynucleotide construct comprising helper virus genes and AAV rep and cap genes are introduced into the expanded producer cells. When the first and second polynucleotide constructs are plasmids, this system may be referred to as a two-plasmid system.

In embodiments of the method disclosed herein, following the expansion phase a first polynucleotide construct comprising a transgene flanked by ITRs, a second polynucleotide construct comprising helper virus genes, and a third polynucleotide construct comprising AAV rep and cap genes are introduced into the expanded producer cells. When the first, second and third polynucleotide constructs are plasmids, this system may be referred to as a three-plasmid system.

In cases where one or more recombinant plasmids are used to manufacture rAAV vectors, the “rAAV genome production plasmid” refers to a plasmid comprising the transgene (operably linked to regulatory sequences) and one or more ITRs intended for packaging into the rAAV, as well as non-rAAV genome components (the plasmid backbone) that are important for cloning and amplification of the plasmid, but are not packaged or encapsidated into rAAV vectors. The term “construct” as used herein refers to a recombinant polynucleotide construct (i.e., a polynucleotide having elements derived from different sources) which may be a plasmid.

The terms “transduce” and “transfect” refer to introduction of a polynucleotide into a host cell or target cell. In embodiments, the host cell is a producer cell, e.g., a HEK293 cell. In embodiments, the rAAV genome production plasmid along with one or more plasmids providing helper virus functions and AAV functions are introduced into the producer cells by transient transfection methods. The transient transfection of producer cells to introduce the first polynucleotide construct comprising a transgene and ITR(s) (e.g., an rAAV genome production plasmid); and optionally a second and/or third polynucleotide constructs providing AAV functions (rep and cap genes), and helper virus functions, can be accomplished by standard transfection methods including for example calcium phosphate coprecipitation, cationic lipid-based transfection, and cationic polymer-based transfection. Cationic lipid-based transfection includes e.g., Lipofectamine (a 3:1 mixture of DOSPA (2,3-dioleoyloxy-N-[2 (sperminecarboxamido)ethyl]-N,N-dimethyl-1-propaniminium trifluoroacetate) and DOPE (1,2-Dioleoyl-sn-glycero-3-phosphoethanolamine). Cationic polymer-based transfection includes, e.g., using linear and/or branched polyethylenimine (PEI), poly-L-lysine, poly-L-arginine, polyamidoamine dendrimers and others. In embodiments the transient transfection of producer cells is performed using a PEI based transfection reagent. The PEI may be a linear or branched polymer. In embodiments, the PEI is a 20-25 kD linear PEI. For example, in embodiments the PEI is jetPEI or PEIpro (available from Polyplus). Additionally, transient transfection of producer cells can be performed using a transfection reagent comprising both cationic lipids and cationic polymers.

rAAV may alternatively be produced in insect cells (e.g., sf9 cells) using baculoviral vectors or in HSV-infected baby hamster kidney (BHK) cells (e.g., BHK21). In both methods, rAAV production is triggered in the host cells, insect cells or mammalian cells, respectively, upon co-infection with two or more recombinant viruses carrying the rAAV genome and one more AAV rep and cap, and helper virus functions required for rAAV replication and packaging.

Producing the rAAV Particles

Generally, a production phase (also referred to as a production step) follows the step of introducing a rAAV genome vector and/or vectors providing helper virus functions and/or AAV functions into the producer cells. In embodiments, rAAV particles are produced by culturing the cells following introduction of the rAAV genome vector for at least about 48 hours (e.g., 47.5, 48, 50, 55, 60, 65, 70, 75, 80, 85, 90, 95, 96, 96.5, or 97 hours). In embodiments, the transfected producer cells are cultured (i.e., the production phase is maintained) for about 72 to about 100 hours, about 90 hours to about 100 hours, about 92 hours to about 98 hours, or about 94 to about 98 hours. In embodiments the production phase is maintained for about 96 hours.

The production phase medium can be any cell culture medium suitable for production of rAAV in the producer cells. In embodiments, the production medium is free of animal products, such as serum. “Free of” in this context means that the medium has undetectable levels of animal products such as serum. In embodiments, the pH of the production medium is reduced compared to the pH of the expansion phase medium. In embodiments, the production medium is maintained at a pH of about 6.8 to about 7.4 (e.g., about 6.8, about 6.9, about 7.0, about 7.1, about 7.2, about 7.3, or about 7.14). In embodiments the pH is maintained at about 6.9 to about 7.3.

In embodiments of the method, the production phase comprises addition of calcium ions to the production phase cell culture medium. Addition of calcium ions to the production medium, also referred to herein as calcium supplementation, comprises adding calcium ions (Ca²⁺) in the form of calcium salts such as CaCl₂). Calcium ions can be added during the production phase at one or more times following introduction of the rAAV genome vector (e.g., post-transfection). For example, calcium ions can be added at one or more times between about 0 hours to about 48 hours from the start of the production phase (i.e., post transfection), e.g., about 1 hour, about 6 hours, about 10 hours, about 12 hours, about 20 hours, about 24, hours about 30 hours, about 36 hours, and/or about 48 hours.

Calcium ions (e.g., CaCl₂) can be added to the production media to achieve a total concentration of calcium ions in the culture medium greater than 0.3 mM and less than 10 mM. In embodiments, calcium ions are added to a total concentration between about 1 mM to about 9 mM, between about 1 mM to about 8 mM, between about 1 mM to about 7 mM, between about 2 mM to about 9 mM, between about 2 mM to about 8 mM, between about 2 mM to about 7 mM, between about 2 mM to about 6 mM, between about 2 mM to about 5 mM, or between about 2 mM to about 4 mM.

In embodiments, the production phase comprises adding one or more of glutamine, a glutamine precursor or an amino acid dipeptide including glutamine to the production phase medium. The one or more of glutamine, glutamine precursor or an amino acid dipeptide including glutamine can be one or more of, e.g., L-alanyl-L-glutamine, L-glutamine, glutamate, glycyl-L-glutamine, glutamine protein hydrolysate, L-glutamic acid, and a glutamine dipeptide. A solution including at least one of glutamine, glutamine precursor or an amino acid dipeptide including glutamine can be added to the production phase culture medium at, e.g., one or more of about 6 hours, about 12 hours, about 24 hours, about 48 hours or about 72 hours post-transfection.

In embodiments, the production phase comprises adding sorbitol to the production phase medium. The sorbitol can be added to the production phase medium at one or more time points during the production phase, for example at about 6 hours, about 12 hours, about 20 hours, about 24 hours, and/or about 48 hours post transfection. In embodiments, the sorbitol is added to the production medium to a concentration of about 50 mM to about 200 mM, or about 80 mM to about 120 mM. In embodiments, the sorbitol is added to the production medium to a concentration of about 100 mM.

In embodiments, the production phase comprises addition of an anti-clumping supplement to the production phase medium. The anti-clumping supplement may be added at one or more time points to the production phase culture media (for example, at one or more of about 6, about 10, about 12, about 20, about 24, about 48 or about 72 hours post-transfection). In embodiments, the anti-clumping supplement comprises dextran sulfate, heparin and/or other sulfated glycosaminoglycans that suppress the aggregation of the producer cells. In embodiments, the anticlumping supplement comprises sodium heparin, which can be added to the media to concentrations of about 25 μg/ml to about 250 μg/ml, for example, about 25 μg/ml, about 50 μg/ml, about 100 μg/ml, about 150 μg/ml, and/or about 200 μg/ml.

In embodiments, anti-clumping supplement is not added to the production phase media, or is only added to the production phase media shortly before the end of the production phase, for example within about 24 hours, about 12 hours, within about 6 hours, within about 3 hours, within about 2 hours, or within about 1 hour of the end of the production phase.

Isolating and Purifying the rAAV

Embodiments of the methods described herein include isolating and purifying the rAAV particles (rAAV full capsids) at the end of the production phase. In embodiments, the rAAV particles can be isolated at about 48 or more hours (e.g., 47.5, 48, 50, 55, 60, 65, 70, 75, 80, 85, 90, 95, 96, 96.5, 97 hours) after introduction of the rAAV vector, and/or helper function and/or AAV rep/cap sequences (in other words after the start of the production phase). For example, the rAAV particles can be isolated at about 90 to about 100 hours, about 92 hours to about 98 hours, or about 94 to about 98 hours after introduction of the rAAV vector, and/or helper function and/or AAV rep/cap sequences. In embodiments, the rAAV particles are isolated (e.g., the cells are lysed) at about 96 hours after the introduction of the rAAV vector, and/or helper function and/or AAV rep/cap sequences. Isolating the rAAV can include multiple steps including, for example, lysing the producer cells resulting in a cellular lysate, clarifying the lysate resulting in a clarified lysate, and subsequent purification steps.

rAAV particles may be retained within producer cells following generation, and methods to release intracellular rAAV vector include physical and chemical disruption, for example, use of detergent, microfluidization and/or homogenization. In embodiments, cell membrane disruption (lysis) and rAAV or AAV particle release (recovery) from cells is achieved using the amphoteric detergent N,N-Dimethyltetradecylamine N-oxide (TDAO) (commercially available as Deviron® C16 from MilliporeSigma Burlington, MA). For example, when recovering rAAV5, AAV5, rAAV2, or AAV2 particles, Deviron® C16 at about 0.1% to about 0.5% (e.g., 0.09, 0.1, 0.2, 0.3, 0.4, 0.5, 0.51%) can be used. In other embodiments, hyperosmotic shock (an increase in external osmolality) is used to lyse the cells and release rAAV or AAV particles. For example, when recovering rAAV8 or AAV8 particles, the producer cells can be subjected to hyperosmotic shock at an incubation duration of about 90 to about 120 minutes (e.g., about 89, 90, 95, 100, 105, 115, 120, 121 minutes) and a NaCl concentration of about 400 mM (e.g., about 399, 400, 401 mM). Concurrently during cell lysis and/or subsequently after cell lysis, a nuclease such as benzonase may be added to degrade contaminating DNA. Typically, the resulting lysate is clarified to remove cell debris and provide a clarified cell lysate. In embodiments, the clarified lysate is subjected to AEX in weak partition mode to separate the full capsids from the empty capsids as described herein.

### EXAMPLES

### Example 1. Use of Anion Exchange Chromatography in Weak Partitioning Mode to Provide High Empty AAV Capsid Removal and Product Yields

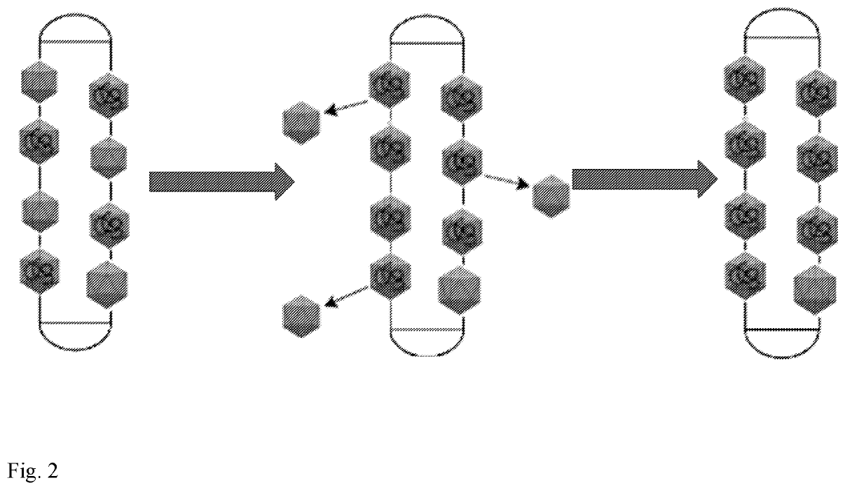

An anion exchange step was developed to maximize full capsid enrichment for AAV-AQP1 (AAV2). The similar properties of the full and empty capsids led to a trade-off between product yield and full capsid ratio which made selecting optimal conditions difficult. Different surfactant types and concentrations were screened to maximize the product yield of the AAV2 vector. Exploring the design space led to weak partitioning mode being identified as an alternative to bind-and-elute mode (FIGS. 1, 2), and conditions that achieved the removal of >50% of the empty capsids in the flow through alone were identified. Coupled with a high NaCl wash an eluate with >80% full capsids was achieved. The results show that a full capsid ratio of >80% as confirmed by Analytical ultracentrifugation (AUC) and a product yield >50% can be robustly achieved using the novel conditions described herein. Understanding the design space and identifying the process failure points meant that the process was reproducible at manufacturing scale.

In the initial design of experiments, parameters and ranges for further optimization were identified from initial AEX platform-fit experiments and are listed in Table 1:

TABLE 1

| Parameter | Range |
|---|---|
| Load challenge (VG/mL) | 5 × 10¹³-1 × 10¹⁴ |
| Eq./Feed NaCl conc. (mM) | 65-90 |
| Wash 2 NaCl conc. (mM) | 100-125 |
| Elution NaCl conc. (mM) | 200-300 |

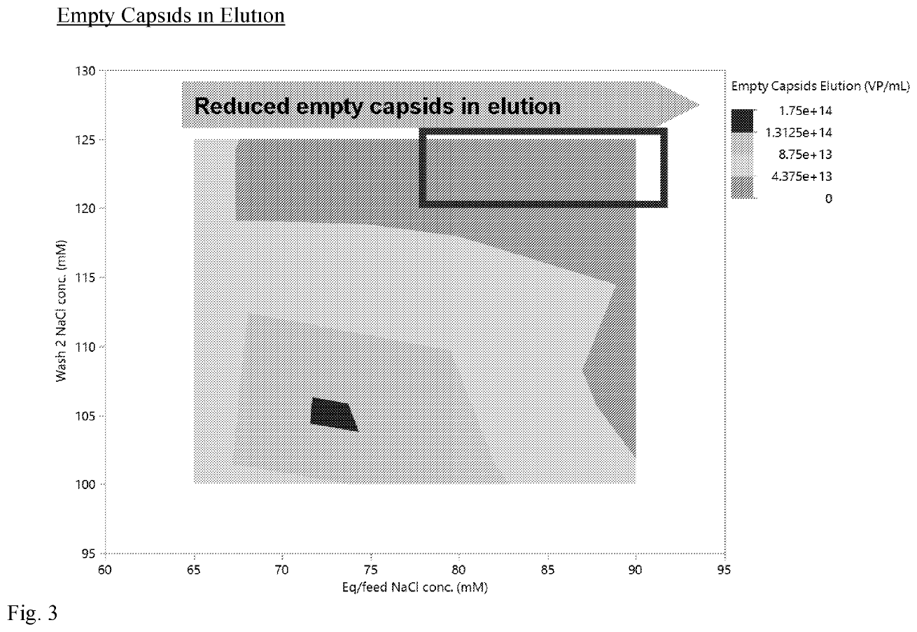

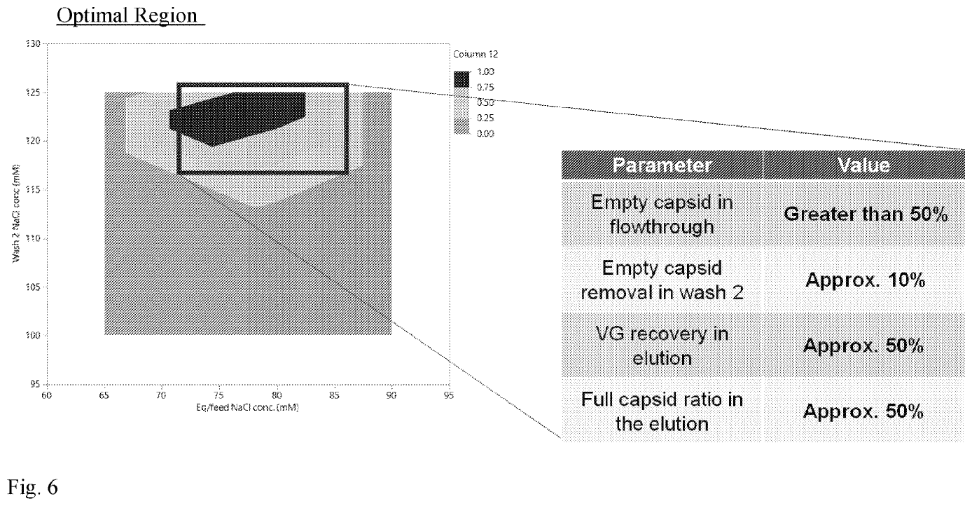

The relationships between wash 2 and eq./feed NaCl concentrations (mM) and (i) empty capsid removal, (ii) full capsid ratio in the elution, and (iii) VG recovery in the elution were analyzed. The results are shown in FIGS. 3-5. Results of a design of experiments study (DOE) (FIG. 6) combined to highlight optimal region within the design space, in line with the desired outcome. Parameters and values from the optimal region based on the wash 2 and eq./feed NaCl concentrations are shown in Table 2:

TABLE 2

| Parameter | Value |
|---|---|
| Empty capsid in flowthrough | Greater than 50% |
| Empty capsid removal in wash 2 | Approx. 10% |
| VG recovery in elution | Approx. 50% |
| Full capsid ratio in the elution | Approx. 50% |

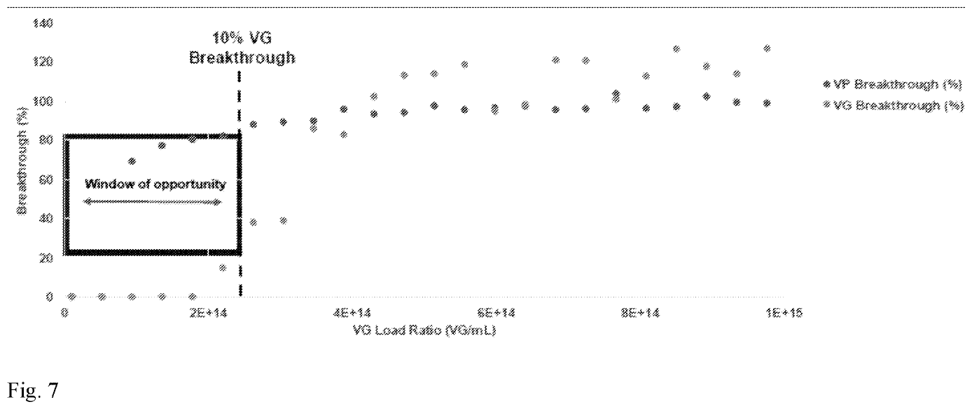

To understand how load ratio affects weak partitioning, the relationship between load ratio, and full capsid (VG) and empty capsid (VP) breakthrough was analyzed. The results (FIG. 7) showed that the effects of weak partitioning are maximized by increasing the load ratio. The parameters and values are shown in Table 3:

TABLE 3

| Parameter | Value |
|---|---|
| Load Ratio (VG/ml.) | 1 × 10¹⁵ |
| Eq./feed NaCl conc. (mM) | 90 |

Referring to FIG. 7, exploiting the window of opportunity means up to 80% of empty capsids breaking through while only 10% of full capsids are lost in the flow through.

Collating the findings, the results shown in FIG. 3 showed that for the largest empty capsid removal, high NaCl concentration was required for both Wash 2 and elution. Increasing the Wash 2 NaCl caused VG losses of up to 30%; 50% VG recovery was still recovered in the elution phase. The parameters and optimized ranges are shown in Table 4:

TABLE 4

| Parameter | Optimized Ranges |
|---|---|
| Load Ratio (VG/ml.) | 1 × 10¹⁴ |
| Eq/Feed NaCl conc. (mM) | 90 |
| Wash 2 NaCl conc. (mM) | 125 |
| Elution NaCl conc. (mM) | 250 |

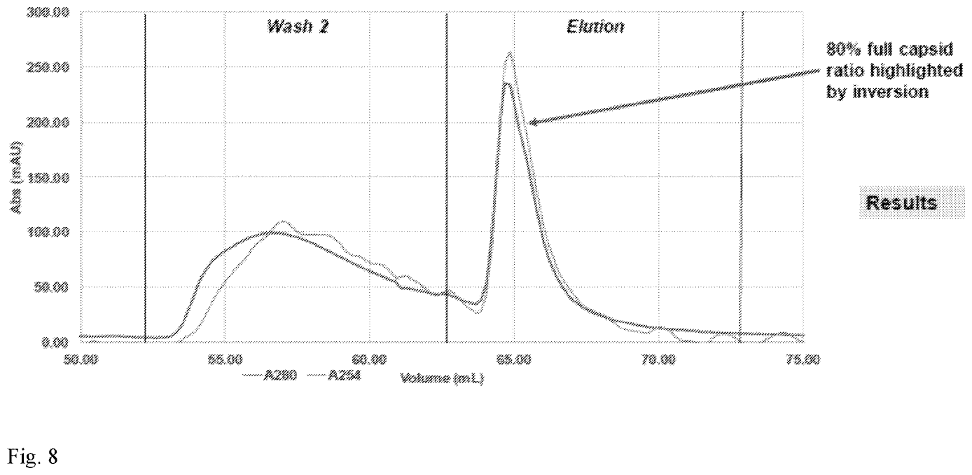

A chromatogram of an optimized AAV-AQP1 AEX step at 2 L scale is shown in FIG. 8, noting an 80% full capsid ratio highlighted by inversion (inversion of the 260 nm and 280 nm UV absorbance traces which typically indicates a high full capsid ratio due to the presence of the encapsidated DNA). The parameters and values are shown in Table 5:

TABLE 5

| Parameter | Value |
|---|---|
| Empty capsids in flowthrough (%) | >40% |
| Full capsids in flowthrough (%) | <1% |
| Full capsids in wash 2 (%) | 33% |
| Empty capsids in wash 2 (%) | 20% |
| VG recovery in elution (%) | 53% |
| Full capsid ratio in elution (%) | 80% |

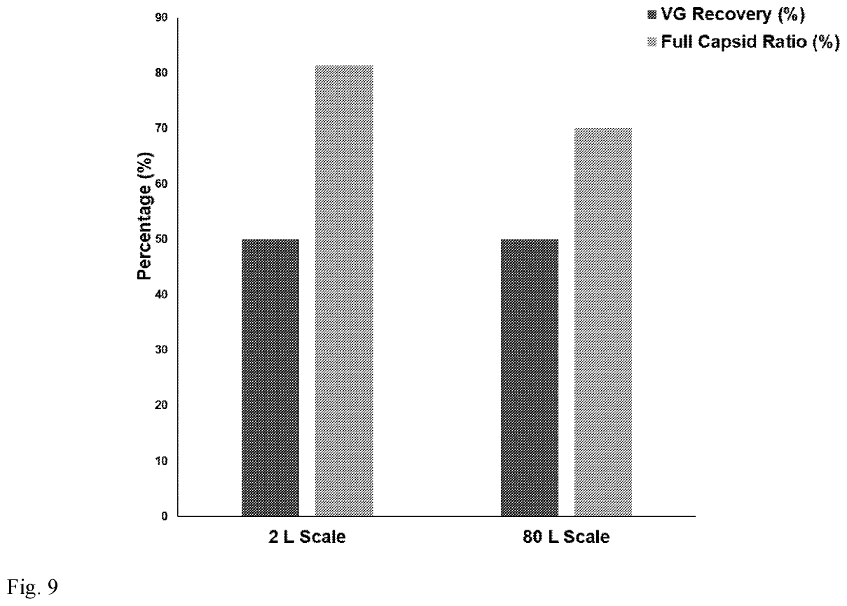

As shown in FIG. 9, the process is scalable from 2 L to 80 L for both the VG recovery and the full capsid ratio.

### Example 2: AEX Weak Partitioning Protocols for Enrichment of Full Capsids and Increasing Yield

The experiments described below demonstrate that the rAAV purification methods described herein are consistent in terms of full capsid ratio and VG recovery, indicating that the process is scalable.

### Materials

### AAV Feed Material

The feed material for the experiments described in this study consisted of adeno associated virus (AAV) capsids of serotype 2 packaged with a transgene encoding for the aquaporin 1 gene.

These capsids were produced via cell culture using HEK293 cells that had been transiently transfected with a triple plasmid complex. After completion of the production phase of the cell culture, the cells were lysed using Triton X-100 to release the AAVs and resulting lysate was treated with Benzonase. The lysate was then clarified using 0.2 μm PES filters.

An initial purification of the AAV capsids was performed using AAVX (ThermoFisher) affinity chromatography resin and an AKTA Avant (Cytiva) chromatography system. This purification was performed by loading the clarified lysate onto an AAVX column with a 5 cm bed height, at a load ratio of 200 mL of clarified lysate per mL of AAVX resin, and a residence time of 1 min. The captured AAV capsids were eluted using an elution buffer consisting of 50 mM glycine, 180 mM NaCl, 0.25% (w/v) polysorbate 80, pH 2.7.

### Chemicals

All chemicals used in these experiments were GMP (good manufacturing practice) grade reagents procured from Merck KGaA.

### Methods

### Anion Exchange Chromatography

All small-scale anion exchange chromatography (AEX) experiments were performed using 1 mL Sartobind Q (Sartorius) AEX membranes and an AKTA Avant (Cytiva) chromatography system.

The buffers for these experiments consisted of 50 mM tris, 0.75% (w/v) polysorbate 80, pH 9 and various concentrations of NaCl as required by the phase of the experiment. The feed for these experiments was AAVX eluate which was conditioned by diluting to the target NaCl concentration with 50 mM tris, 0.75% (w/v) polysorbate 80, pH 9.0. Table 6 describes the method that was used for the AEX experiments in this study.

TABLE 6
Overview of method used for AEX experiments

| | | Membrane | |
|---|---|---|---|
| | | volumes | Flow rate |
| Phase | Buffer | [MV] | [MV/min] |
| Pre | 50 mM Tris, 1M NaCl, 0.75% | 15 | 10 |
| equilibration | polysorbate 80, pH 9.0 | | |
| Equilibration | See experimental details | 15 | 10 |
| Sample | Conditioned feed | | 10 |
| application | | | |
| Column wash 1 | See experimental details | 10 | 10 |
| Column wash 2 | See experimental details | 10 | 10 |
| Elution | See experimental details | 15 | 10 |
| Strip | 50 mM Tris, 1M NaCl, 0.75% | 10 | 10 |
| | polysorbate 80, pH 9.0 | | |
| CIP | 0.5M NaOH | 15 | 10 |
| Equilibration | 50 mM Tris, 0.75% | 10 | 10 |
| | polysorbane 80, pH 9.0 | | |

### Design of Experiments

A DoE study was performed to evaluate the impact of the load, wash and elution phase NaCl concentrations, and the load ratio on the full capsid ratio and the vector genome (VG) recovery. This study consisted of 16 runs and Table 7 describes the parameter values that were tested in each experiment. Samples of the flow-through, wash 2 and eluate were collected from each run and analyzed for their vector genome and vector particle concentration.

TABLE 7
Design of experiments study that was performed to evaluate the impact
of the tested process variables on full capsid purification

| | Eq./feed | Wash 2 | Elution | |
|---|---|---|---|---|
| | NaCl conc. | NaCl conc. | NaCl conc. | Load ratio |
| Run | [mM] | [mM] | [mM] | [VG/mL] |
| 1 | 80 | 100 | 240 | 7.50 × 10¹³ |
| 2 | 80 | 110 | 250 | 5.00 × 10¹³ |
| 3 | 80 | 125 | 300 | 1.00 × 10¹⁴ |
| 4 | 80 | 110 | 250 | 5.00 × 10¹³ |
| 5 | 72.5 | 105 | 200 | 1.00 × 10¹⁴ |
| 6 | 72.5 | 122.5 | 200 | 7.50 × 10¹³ |
| 7 | 72.5 | 105 | 300 | 7.50 × 10¹³ |
| 8 | 90 | 125 | 210 | 1.00 × 10¹⁴ |
| 9 | 90 | 107.5 | 200 | 5.00 × 10¹³ |
| 10 | 90 | 125 | 220 | 5.00 × 10¹³ |
| 11 | 90 | 100 | 300 | 1.00 × 10¹⁴ |
| 12 | 90 | 100 | 300 | 5.00 × 10¹³ |
| 13 | 90 | 115 | 280 | 7.50 × 10¹³ |
| 14 | 65 | 117.5 | 260 | 1.00 × 10¹⁴ |
| 15 | 65 | 100 | 200 | 5.00 × 10¹³ |
| 16 | 65 | 125 | 290 | 5.00 × 10¹³ |

### Breakthrough Experiment

A breakthrough experiment was performed to further evaluate the impact of load ratio on the AEX process. The AEX membrane was loaded to a load ratio of 1×10¹⁵ VG/mL with feed that had been conditioned to an NaCl concentration of 90 mM. The flow-through was fractionated into 1 mL fractions and the resulting fractions were analyzed to determine their VG and VP concentrations.

### Confirmation and Scale Up Experiments

The optimized process was confirmed by performing a purification on a 10 mL Sartobind Q module. The method used for this run is detailed in Table 8. Samples of the flow through, column wash 2, and elution were analyzed for their VG and VP concentrations. Additionally, a sample of the elution was analyzed by analytical ultracentrifugation and qPCR to confirm the full capsid ratio and VG recovery.

TABLE 8
Method used to confirm the optimized AEX process.

| | | Membrane | |
|---|---|---|---|
| | | volumes | Flow rate |
| Phase | Buffer | [MV] | [MV/min] |
| Pre | 50 mM Tris, 1M NaCl, 0.75% | 15 | 10 |
| equilibration | polysorbate 80, pH 9.0 | | |
| Equilibration | 50 mM Tris, 90 mM NaCl, 0.75% | 15 | 10 |
| | polysorbate 80, pH 9.0 | | |
| Sample | Feed conditioned to 90 mM NaCl | | 10 |
| application | | | |
| Column | 50 mM Tris, 90 mM NaCl, 0.75% | 10 | 10 |
| wash 1 | polysorbate 80, pH 9.0 | | |
| Column | 50 mM Tris, 125 mM NaCl, 0.75% | 10 | 10 |
| wash 2 | polysorbate 80, pH 9.0 | | |
| Elution | 50 mM Tris, 250 mM NaCl, 0.75% | 15 | 10 |
| | polysorbate 80, pH 9.0 | | |
| Strip | 50 mM Tris, 1M NaCl, 0.75% | 10 | 10 |
| | polysorbate 80, pH 9.0 | | |
| CIP | 0.5M NaOH | 15 | 10 |
| Equilibration | 50 mM Tris, 0.75% | 10 | 10 |
| | polysorbate 80, pH 9.0 | | |

To demonstrate scalability, the process was scaled up to a 75 mL Sartobind® Q membrane. This run was performed using the same method detailed in Table 8 and a sample of the elution was analyzed by analytical ultracentrifugation and qPCR to determine its full capsid ratio and VG recovery.

### Analytical Methods

The vector genome concentration in the samples produced in this study was determined by qPCR using primers and probes specific to a region of the AQP1 transgene. The vector particle concentration was determined using the Gyrolab® AAVX Titer Kit (Gyros Protein Technologies).

During the DoE and breakthrough experiments, the empty capsid ratio was estimated by dividing the vector genome concentration by the vector particle concentration. In the confirmation and scale up experiments the full capsid ratio was measured by analytical ultracentrifugation using an Optima AUC (Beckman).

### Results

### DoE

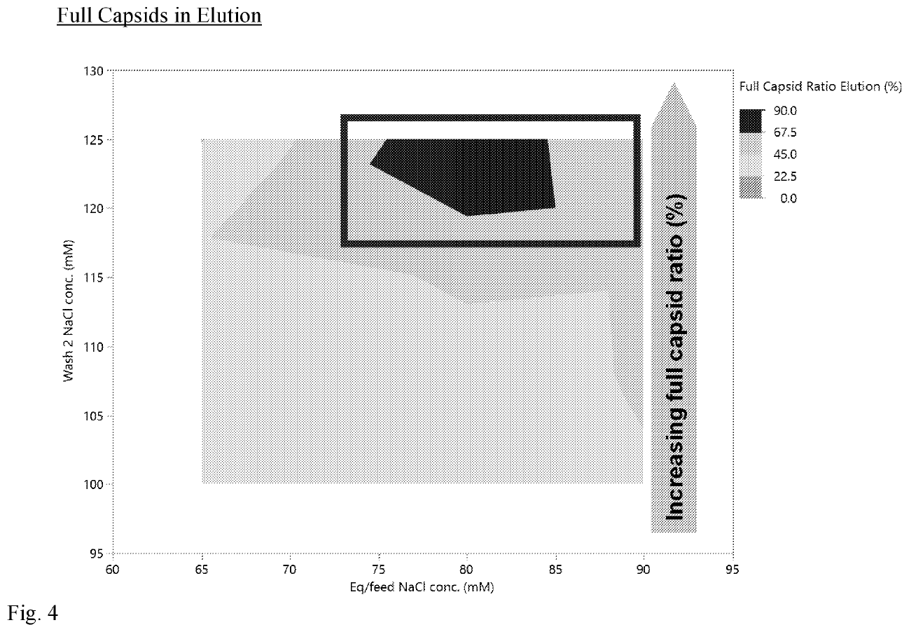

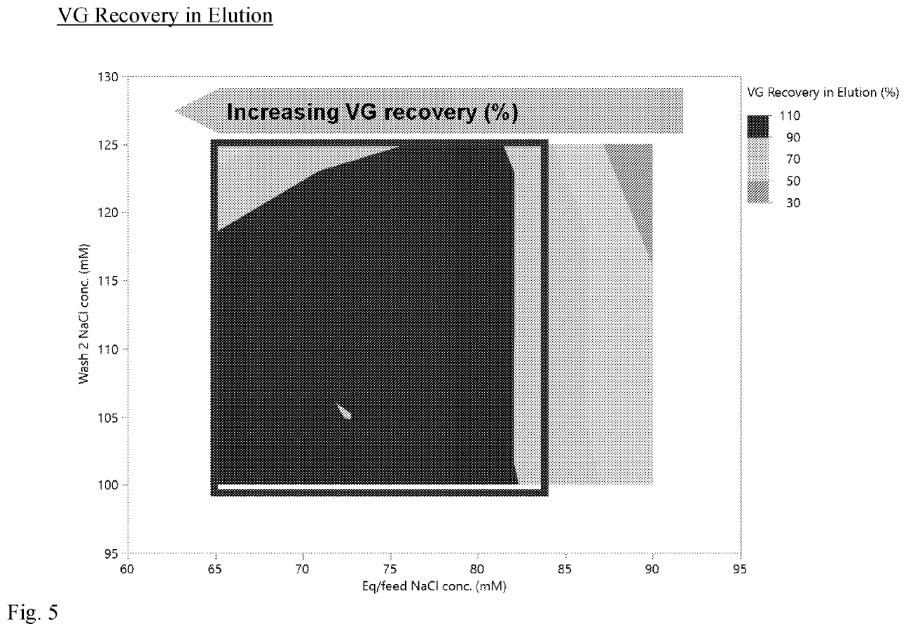

The design space for the AEX process was investigated by performing a design of experiments study with the objective of identifying conditions that maximize full capsid ratio and VG recovery. The variables that were explored in this study were the feed NaCl concentration, the wash 2 NaCl concentration, the elution NaCl concentration, and the load ratio. The ranges that were tested for these variables are detailed in Table 9. The results of these experiments are detailed in Table 10 while the trends observed in this dataset are illustrated in FIG. 3, FIG. 4, and FIG. 5.

TABLE 9
Ranges that were tested for each variable
of the design of experiments study

| Parameter | Range |
|---|---|
| Load challenge (VG/mL) | 5 × 10¹³-1 × 10¹⁴ |
| Eq./feed NaCl conc. (mM) | 65-90 |
| Wash 2 NaCl conc. (mM) | 100-125 |
| Elution NaCl conc. (mM) | 200-300 |

TABLE 10
Results of the design of experiments study performed
to explore the design space of the AEX process.

| | | | | Full | VG | VP |
|---|---|---|---|---|---|---|
| Eq/feed | Wash 2 | Elution | | Capsid | Recovery | Recovery |
| NaCl | NaCl | NaCl | Load | Ratio | in | in |
| conc. | conc | conc | Ratio | Elution | Elution | Elution |
| (mM) | (mM) | (mM) | (VG/mL) | (%) | (%) | (%) |
| 80 | 100 | 240 | 7.50E+13 | 30 | 100 | 19.3 |
| 80 | 110 | 250 | 5.00E+13 | 64 | 100 | 55 |
| 80 | 125 | 300 | 1.00E+14 | 87.3 | 100 | 64 |
| 80 | 110 | 250 | 5.00E+13 | 4.8 | 100 | 138.7 |
| 72.5 | 105 | 200 | 1.00E+14 | 41.6 | 100 | 0 |
| 72.5 | 122.5 | 200 | 7.50E+13 | 60.3 | 97.3 | 0 |
| 72.5 | 105 | 300 | 7.50E+13 | 16.38 | 79.25 | 49.56 |
| 90 | 125 | 210 | 1.00E+14 | 49.6 | 31.88 | 10.37 |
| 90 | 107.5 | 200 | 5.00E+13 | 47.16 | 51.63 | 4.71 |
| 90 | 125 | 220 | 5.00E+13 | 38.05 | 28.46 | 15.9 |
| 90 | 100 | 300 | 1.00E+14 | 49.4 | 58.29 | 1.11 |
| 90 | 100 | 300 | 5.00E+13 | 36.15 | 56.39 | 1.69 |
| 90 | 115 | 280 | 7.50E+13 | 47.72 | 52.24 | 6.45 |
| 65 | 117.5 | 260 | 1.00E+14 | 44 | 94.21 | 0 |
| 65 | 100 | 200 | 5.00E+13 | 26.28 | 109.8 | 46.42 |
| 65 | 125 | 290 | 5.00E+13 | 21.67 | 62.73 | 105.53 |

The feed for these experiments had an estimated full capsid ratio of less than 10%. Table 10 shows that some of the tested conditions resulted in a significant increase of estimated full capsid ratio (up to 87%). FIG. 3 shows that high eq./feed and wash 2 NaCl concentrations lead to reduced empty capsids in the elution pool. This is because the higher NaCl concentrations in the feed prevent empty capsids from binding to the AEX membrane. Similarly higher NaCl concentrations in the column wash 2 will cause the elution of the empty capsids during this phase, removing them from the later elution pool.

FIG. 4 shows how these effects translate into a higher full capsid ratio in the elution pool. As the empty capsids are prevented from binding or are washed off the column, the resulting elution pool has a high full capsid ratio. The impact of the increasing feed and column wash 2 NaCl concentration is shown in FIG. 5. Despite the full capsids having a stronger interaction with the AEX membrane, some of these full capsids are lost to the flow-through and wash as the feed and wash 2 NaCl concentrations increase and disrupt these interactions. FIG. 6 combines the trends seen in these experiments to highlight the area where a high full capsid ratio and a high VG recovery can be achieved.

### Breakthrough Experiment

It was observed on the DoE study above that empty capsids flowing through the membrane at higher feed NaCl concentrations without binding to the Sartobind® Q AEX membrane. This effect was further investigated by performing a breakthrough experiment. This experiment was performed with a feed NaCl concentration of 95 mM to maximize this effect and the membrane was loaded to 1×10¹⁵ VG/mL to ensure that capsids broke through into flow-through.

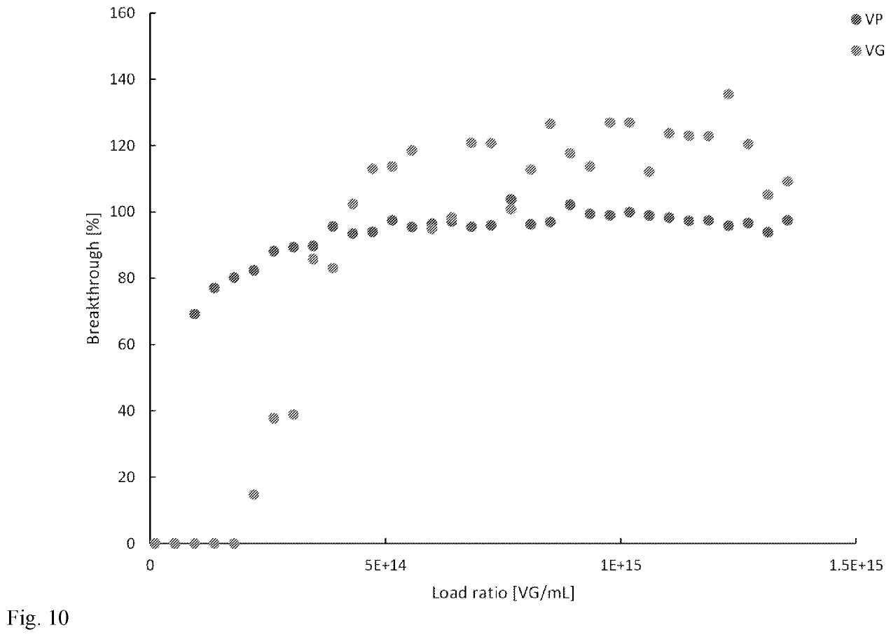

The samples of flow-through were analyzed to determine their VG and VP concentrations and the results are shown in FIG. 10. These results show that under these feed conditions, vector particles start breaking through almost immediately after the start of the experiment. However, as vector genomes do not breakthrough until a load ratio of approximately 2.5×10¹⁴ VG/mL, it can be concluded that the vector particles breaking through are empty capsids.

The results also show that the vector genomes undergo a gradual breakthrough. Typically a sharp breakthrough profile would be expected, indicating that the chromatography matrix had reached saturation. However, this gradual breakthrough indicates that weak partitioning may be taking place. Under weak partitioning, the full capsids begin to displace empty capsids that have bound to the chromatography membrane due to the stronger charged interaction that the full capsids have with the membrane. This has the benefit of further increasing the full capsid ratio of the product that is eventually eluted off the membrane.

### Confirmation Run and Scale Up

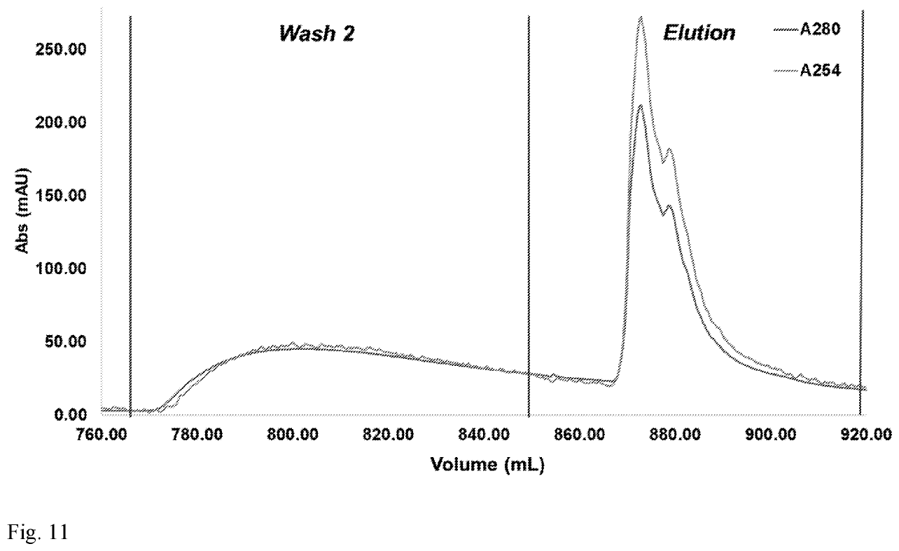

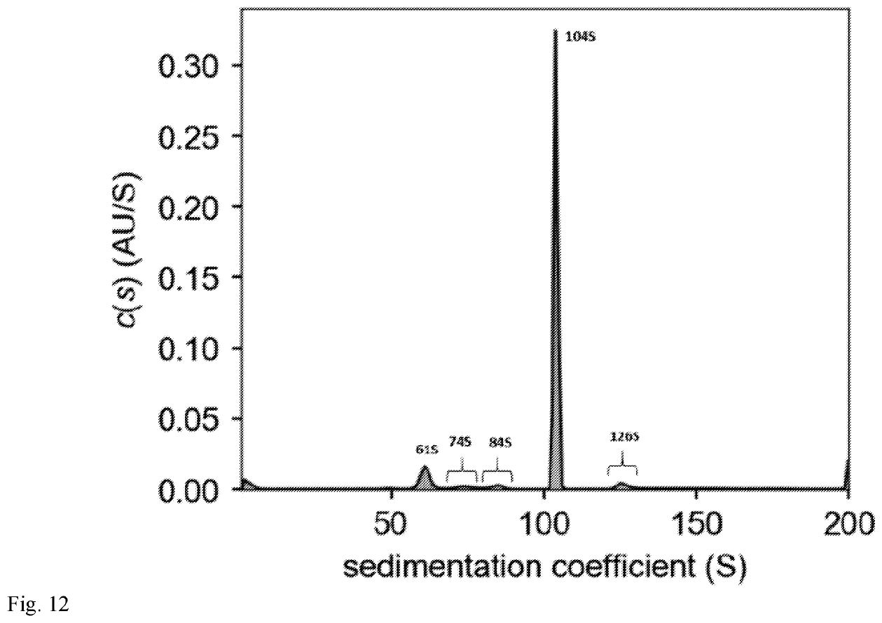

The identified process conditions were confirmed by performing a run at a larger scale using a 10 mL Sartobind® Q and the process conditions detailed in Table 8. An excerpt of the chromatogram for this run, focusing on the column wash 2 and elution phases, is shown in FIG. 11. This figure shows inversion of the 260 nm and 280 nm UV absorbance traces which typically indicates a high full capsid ratio due to the presence of the encapsidated DNA. The eluate from this run was analyzed by analytical ultracentrifugation to confirm the full capsid ratio. The full capsid ratio was found to increase from 31% in the feed to 81.5% in the eluate. The corresponding AUC trace for the eluate is shown in FIG. 12.

A further run was performed on a 75 mL Sartobind® Q to confirm the scalability of the process. The results of 10 mL and 75 mL runs are compared in FIG. 9. This figure shows that the process is consistent in terms of full capsid ratio and VG recovery, indicating that the process is scalable.

### Example 3. Identifying Parameters and Ranges from Initial AEX Platform and Additional Experiments

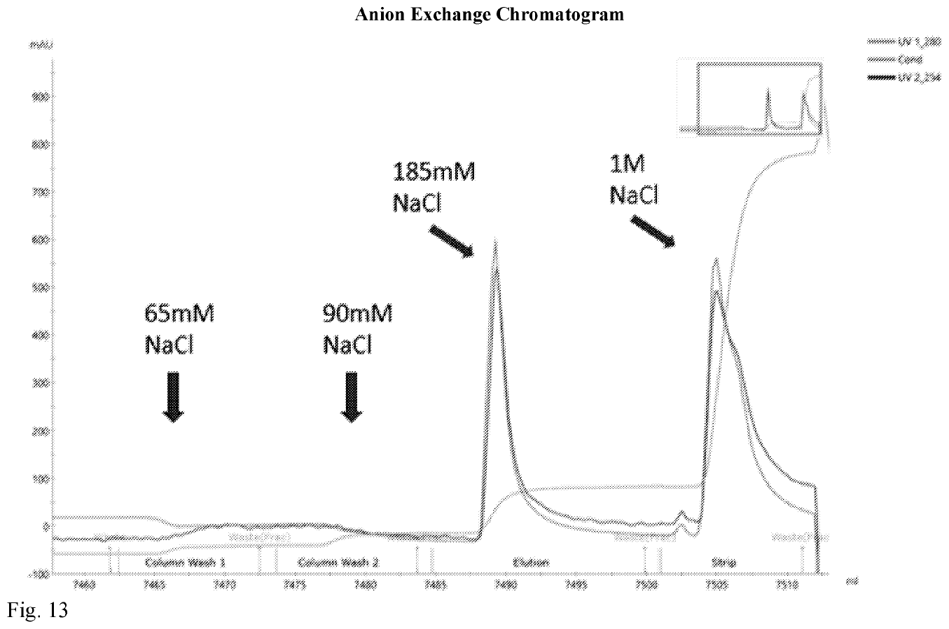

Platform-fit experiments were performed. Shown in FIG. 13 is a chromatogram of the initial AEX chromatography run through. The conditions used in the AEX step are indicated below:

| Parameter | Value |
|---|---|
| Target Load | 6E13 |
| Challenge (VG/mL) | |
| Step | Buffer |
| Preequilibration | 50 mM tris, 1M NaCl, 0.75% (w/v) |
| | polysorbate 80, pH 9.0 |
| Equilibration | 50 mM tris, 65 mM NaCl, 0.75% (w/v) |
| | polysorbate 80, pH 9.0 |
| Column Wash 1 | 50 mM tris, 65 mM NaCl, 0.75% (w/v) |
| | polysorbate 80, pH 9.0 |
| Column Wash 2 | 50 mM Tris, 90 mM NaCl, pH 9 |
| Elution | 50 mM tris, 185 mM NaCl, 0.75% (w/v) |
| | polysorbate 80, pH 9.0 |
| Strip | 50 mM tris, 1M NaCl, 0.75% (w/v) |
| | polysorbate 80, pH 9.0 |
| Step | Step Length |
| Preequilibration | 15 CV |
| Equilibration | 15 CV |
| Column Wash 1 | 10 CV |
| Column Wash 2 | 10 CV |
| Elution | 15 CV |
| Strip | 10 CV |

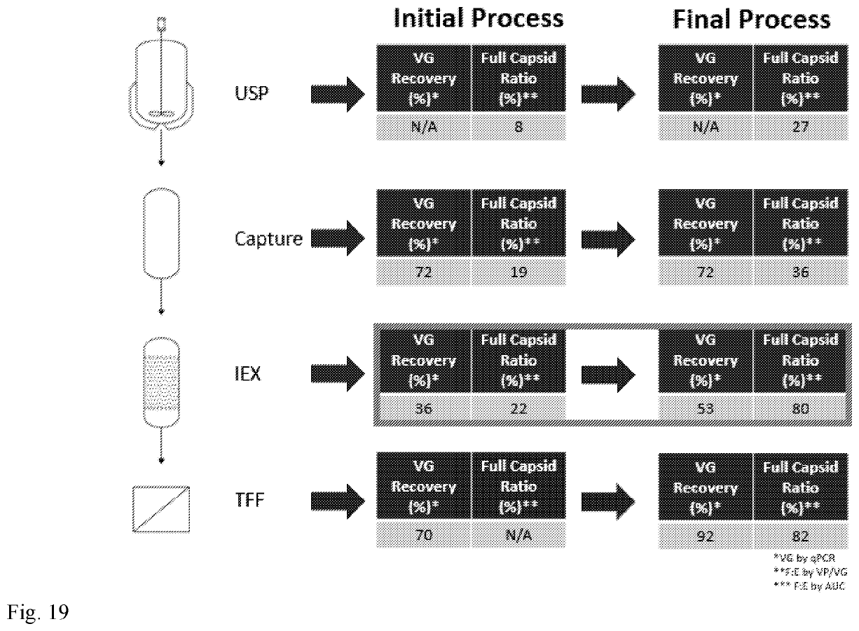

The parameters and ranges shown below in Table 11 were identified from the initial AEX platform-fit experiments (FIG. 13). These initial experiments are referred to as “Initial Process” in FIG. 19 which indicates the VG recovery and full capsid % values after each of: USP, capture chromatography, ion exchange chromatography (IEX), and tangential flow filtration (TFF) in the Initial Process. These values are compared to those obtained from the inventive optimized process described herein (see “Final Process” in FIG. 19).

TABLE 11
Custom DOE conditions

| Parameter | Range |
|---|---|
| Load challenge (VG/mL) | 5 × 10¹³-1 × 10¹⁴ |
| Feed full capsid ratio (%) | <10 |
| Eq./feed NaCl conc. (mM) | 65-90 |
| Wash 2 NaCl conc. (mM) | 100-125 |
| Elution NaCl conc. (mM) | 200-300 |

See FIGS. 3-5 for the following relationships between parameters and empty capsids, full capsids, and VG recovery % in the elution:
FIG. 3: Relationship between wash 2 and eq./feed NaCl concentrations (mM), and the empty capsids present in the elution (VP/mL)
FIG. 4: Relationship between wash 2 and eq./feed NaCl concentrations (mM), and the full capsid ratio (%) in the elution
FIG. 5: Relationship between wash 2 and eq./feed NaCl concentrations (mM), and the VG recovery (%) in the elution

FIG. 6 shows the optimal region based on the wash 2 and eq/feed NaCl concentrations (mM) of FIGS. 1-3. FIGS. 1 and 2 show the weak partitioning process, where the empty capsids are replaced by full capsids due to the environment created. The charge of empty and full AAV capsids above and below the pI is shown. (Cytiva Life Sciences Marlborough, Massachusetts 2022. Enhanced AAV downstream processing).

An additional breakthrough experiment was performed to identify whether at different feed and equilibration conductivities weak partitioning is exploited at different rates, when increasing the load challenge.

### Methods and Materials

Run 1: Feed material and equilibration buffer at a conductivity of 90 mM NaCl.
Run 2: Feed material and equilibration buffer at a conductivity of 80 mM NaCl.

A 0.08 mL Sartobind Q was loaded with AAV-AQP1 neutralised AAVX eluate to 4E14 VG/mL, and the flowthrough samples were fractionated and analysed using the qPCR for VG titre and the Gyrolab for VP titre.

TABLE 12
Load challenge of AAQ-AQP1 AAVX material

| Parameter | Value |
|---|---|
| Load Challenge (VG/mL) | 4E14 |

TABLE 13
The buffers used in both breakthrough curve experiments

| Step | Buffer |
|---|---|
| Equilibration | Run 1: 50 mM Tris, 90 mM NaCl, 0.75% |
| | Tween 80, pH 9 |
| | Run 2: 50 mM Tris, 80 mM NaCl, 0.75% |
| | Tween 80, pH 9 |
| Column Wash 1 | Run 1: 50 mM Tris, 90 mM NaCl, 0.75% |
| | Tween 80, pH 9 |
| | Run 2: 50 mM Tris, 80 mM NaCl, 0.75% |
| | Tween 80, pH 9 |
| Column Wash 2 | 50 mM Tris, 125 mM NaCl, pH 9 |
| Elution | 50 mM Tris, 250 mM NaCl, pH 9 |

TABLE 14
The length of the steps in the chromatography
runs. Method for both run one and run two.

| Step | Step Length |
|---|---|
| Preequilibration | 10 CV |
| Equilibration | 15 CV |
| Sample Application | 0.5 mL fractions |
| Column Wash 1 | 15 CV |
| Column Wash 2 | 15 CV |
| Elution | 15 V |

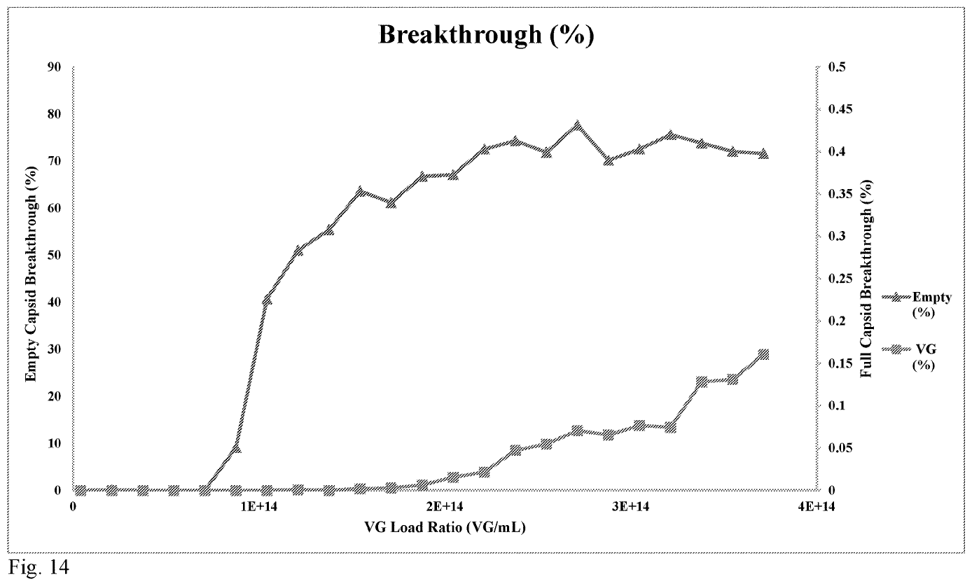

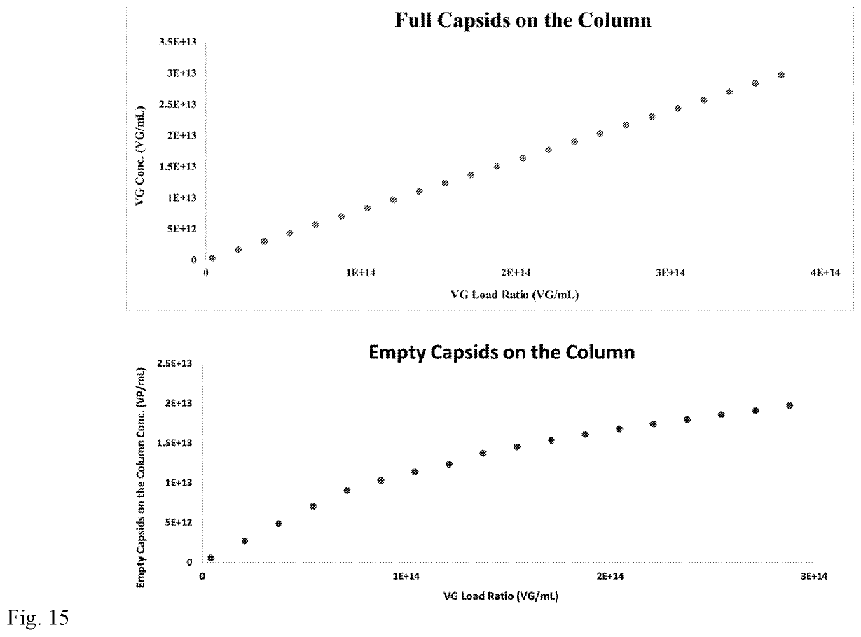

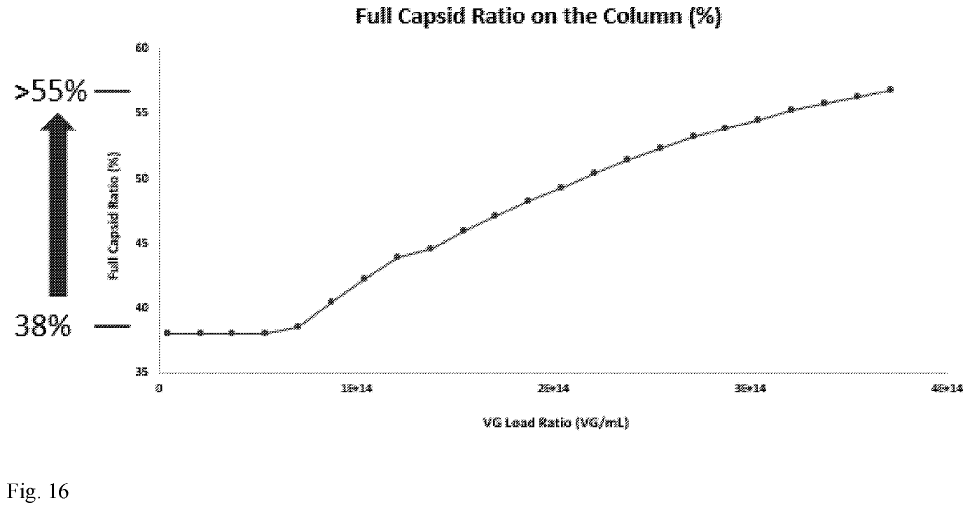

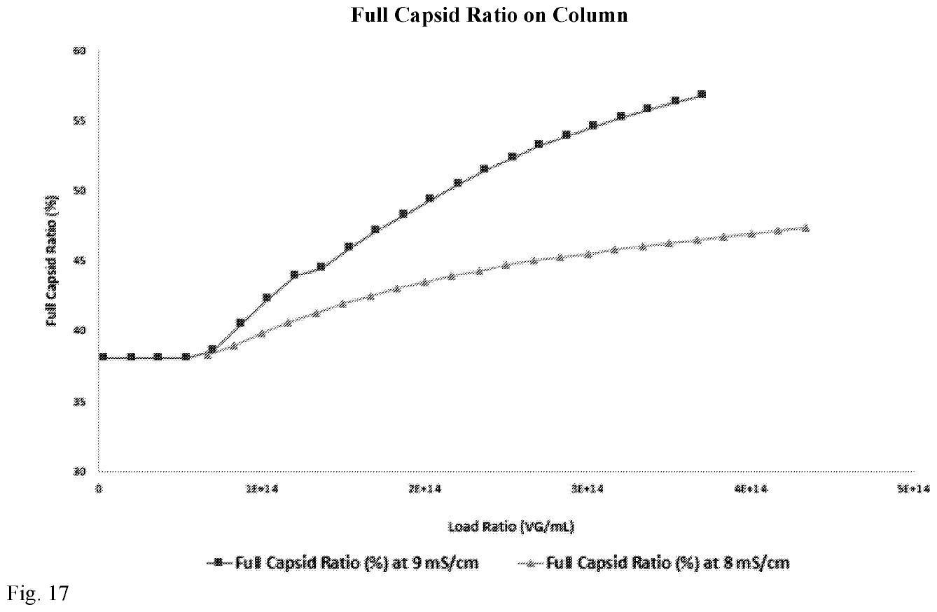

From FIG. 14 it can be seen that when calculating the breakthrough curves for both the empty capsids and the full capsids, the targeted conductivity of 9 mS/cm seems to favor the binding of the full capsids over the empty capsids. To examine what is happening on the column, the VG concentration on the column as the VG load challenge is increased to 4E14 vg/ml was examined (FIG. 15, top graph), and the empty capsid ratio on the column as the VG load challenge is increased to 4E14 VG/mL was examined (FIG. 15, bottom graph). To determine the impact on the full capsid ratio on the column, full capsid ratio on the column as VG load challenged increased was analyzed. As shown in FIG. 16, an increase was observed from 38% to >55% prior to any form of wash or elution steps. Similar to what is shown in FIG. 14, when looking at FIG. 17, it is clear that loading at the targeted 9 mS/cm allows a greater level of enrichment than at the targeted 8 mS/cm, suggesting feed and equilibration feed conductivities are important when trying to exploit weak partitioning and maximize enrichment.

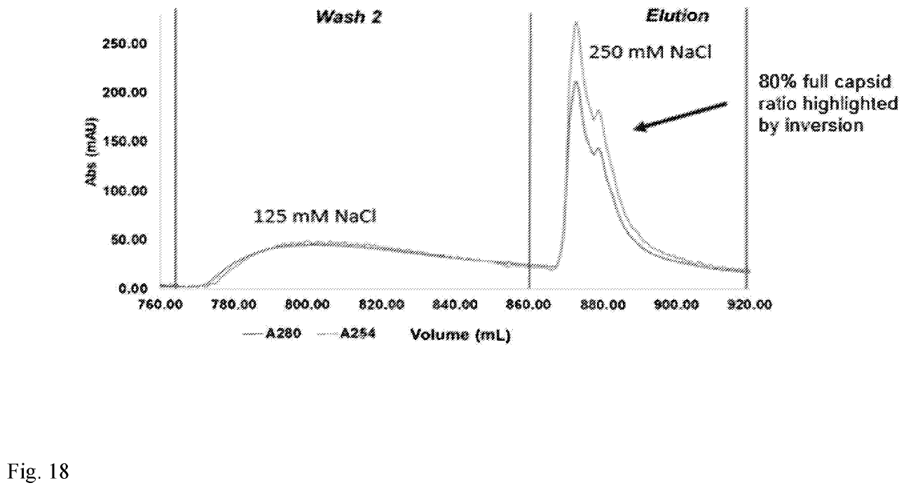

See FIG. 18 for a chromatogram of the optimized AAV-AQP1 AEX step at 20 L scale confirmation run. As shown in this figure, an 80% full capsid ratio was obtained. The parameters and values from this confirmation run are shown below in Table 15. Referring again to FIG. 19, the differences between the “Initial Process” values and the “Final Process” values are shown.

TABLE 15

| Parameter | Value |
|---|---|
| Empty capsids in flowthrough (%) | >40% |
| Full capsids in flowthrough (%) | <1% |
| Full capsids in wash 2 (%) | 33% |
| Empty capsids in wash 2 (%) | 20% |
| VG recovery in elution (%) | 53% |
| Full capsid ratio in elution (%) | 80% |

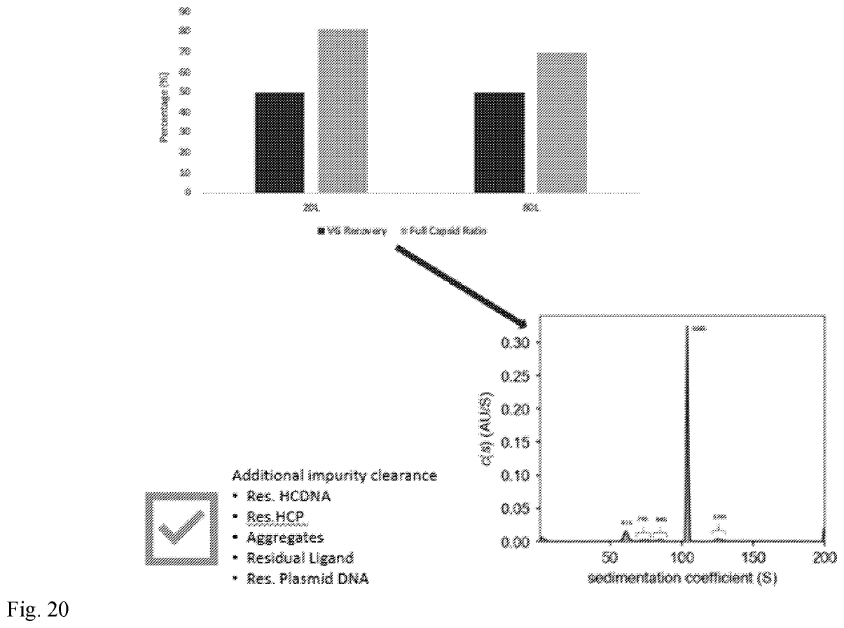

Regarding scale up to 20 L and 80 L batches, a comparison between the full capsid ratio and VG recovery of a 20 L and 80 L batch is shown in FIG. 20. This figure also includes an AUC result confirming the elution peak in FIG. 18 contains 80% full capsids.

We claim:

1. A method of enriching recombinant adeno-associated virus (rAAV) full capsids in a mixture of full and empty capsids, the method comprising the steps of:
(i) providing a solution comprising rAAV full and empty capsids;
(ii) equilibrating an anion exchange (AEX) column or membrane; and
(iii) subjecting the solution comprising rAAV full and empty capsids to weak partition mode AEX chromatography to separate the empty capsids from the full capsids resulting in an AEX eluate enriched for full capsids.

2. The method of claim 1, wherein the solution is an affinity chromatography eluate and step (iii) comprises applying the affinity chromatography eluate to the equilibrated AEX column or membrane, washing the AEX column or membrane at least once, and eluting rAAV full capsids from the AEX column or membrane, wherein the weak partition mode results in full capsids displacing bound empty capsids from the AEX column or membrane and the empty capsids flowing through the AEX column or membrane to produce AEX flow-through and the full capsids remaining bound to the AEX column or membrane until elution.

3. The method of claim 2, wherein the affinity chromatography eluate is diluted to a target salt concentration prior to being subjected to the equilibrated AEX column or membrane.

4. The method of claim 3, wherein the target salt concentration is in a range of about 85 mM to about 95 mM.

5. The method of claim 4, wherein the target salt concentration is about 90 mM and the salt is NaCl.

6. The method of claim 1, wherein the AEX column or membrane is equilibrated prior to loading with the solution comprising rAAV full and empty capsids with an equilibration buffer comprising about 50 mM Tris, about 85 mM to about 95 mM NaCl, and greater than about 0.5% (w/v) polysorbate 80, wherein the equilibration buffer is at a pH of about 9.0.

7. The method of claim 6, wherein the equilibration buffer comprises about 50 mM Tris, about 90 mM NaCl, and about 0.75% polysorbate 80, wherein the equilibration buffer is at a pH of about 9.0.

8. The method of claim 2, wherein washing the AEX column or membrane at least once comprises a first wash buffer comprising (i) about 50 mM tris; (ii) about 85 mM to about 95 mM NaCl, and (iii) greater than about 0.5% (w/v) polysorbate 80, wherein the first wash buffer is at about pH 9.0.

9. The method of claim 8, wherein the first wash buffer comprises (i) about 50 mM tris; (ii) about 90 mM NaCl, and (iii) about 0.75% polysorbate 80, wherein the first wash buffer is at about pH 9.0.

10. The method of claim 2, wherein washing the AEX column or membrane at least once further comprises a second wash with a second wash buffer comprising (i) about 50 mM tris; (ii) about 100 mM to about 125 mM NaCl, and (iii) greater than about 0.5% (w/v) polysorbate 80, wherein the second wash buffer is at about pH 9.0.

11. The method of claim 10, wherein the second wash buffer comprises (i) about 50 mM tris; (ii) about 125 mM NaCl, and (iii) about 0.75% polysorbate 80, wherein the second wash buffer is at about pH 9.0.

12. The method of claim 2, wherein the full capsids are eluted from the AEX column or membrane with an elution buffer comprising (i) about 50 mM tris; (ii) about 125 mM-250 mM NaCl, and (iii) greater than about 0.5% (w/v) polysorbate 80, wherein the elution buffer is at about pH 9.0.

13. The method of claim 12, wherein the elution buffer comprises (i) about 50 mM tris; (ii) about 250 mM NaCl, and (iii) about 0.75% polysorbate 80, wherein the elution buffer is at about pH 9.0.

14. The method of claim 1, wherein the AEX chromatography column or membrane comprises a high flow rate adsorptive membrane.

15. The method of claim 14, wherein the high flow rate adsorptive membrane comprises Sartobind® Q chromatography membranes.

16. The method of claim 1, further comprising the step of subjecting the AEX eluate or a sample thereof to analytical ultracentrifugation to quantify full capsid enrichment.

17. The method of claim 1, wherein the enriched full capsids comprise AAV serotype 2 capsid proteins and a polynucleotide sequence comprising a transgene.

18. The method of claim 17, wherein the transgene is aquaporin 1 (AQP1).

19. The method of claim 1, wherein the enriched full capsids comprise greater than 80% full capsids.

20. The method of claim 19, wherein the enriched full capsids comprise about 81.5% full capsids.

21. The method of claim 19, wherein the enriched full capsids comprise greater than 90% full capsids.

22. The method of claim 1, wherein the enriched full capsids comprise greater than 95% full capsids.

23. The method of claim 1, further comprising the step of subjecting the AEX eluate comprising enriched full capsids to tangential flow filtration resulting in a purified preparation of full capsids.

24. A method for isolating and enriching rAAV full capsids in a mixture of full and empty capsids, the method comprising the steps of:
(i) isolating a mixture of rAAV full and empty capsids from viral production cells by lysing the cells and clarifying the resulting lysate;
(ii) subjecting the clarified lysate to affinity chromatography resulting in an affinity chromatography eluate; and
(iii) subjecting the affinity chromatography eluate to weak partition mode AEX chromatography to separate empty capsids from full capsids resulting in an AEX eluate comprising enriched full capsids.

25. The method of claim 24, wherein the cells are mammalian cells.

26. The method of claim 25, wherein the cells are cultured in suspension.

27. The method of claim 26, wherein the cells are cultured in a shaker flask, a spinner flask, a cellbag or a bioreactor.

28. A population of full capsids enriched by a method according to any one of claims 1 to 27.

29. A pharmaceutical composition comprising the population of full capsids of claim 28.
